(19) United States
(12) Patent Application Publication
(10) Pub. No.: US 2009/0306471 A1
(43) Pub. Date: Dec. 10, 2009

(54) ACCESSING A BODY CAVITY THROUGH THE URINARY TRACT

(75) Inventor: Matthew T. Gettman, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/296,975

(22) PCT Filed: Apr. 16, 2007
(86) PCT No.: PCT/US2007/066711

§ 371 Date: Jul. 16, 2009

Related U.S. Application Data
(60) Provisional application No. 60/793,000, filed on Apr. 18, 2006.

(51) Int. Cl. A61B 17/32; A61B 1/00

(52) U.S. Cl. 600/104; 606/170

(57) ABSTRACT

Some embodiments of the system described herein provide transvesical access to a body cavity (e.g., the peritoneum, the bladder, the ureter, the renal pelvis, or the retroperitoneum). For example, such a transvesical approach may provide access to the peritoneal cavity through an opening formed in the bladder wall or other structures in the urinary tract (e.g., ureter, renal pelvis, or the like). Thus, the transvesical approach through the bladder wall permits a surgeon to examine, conduct surgical or therapeutic procedures, or a combination thereof inside peritoneal cavity. Further, the transvesical access to the peritoneal cavity or another body cavity provides the opportunity to use flexible or rigid endoscopes, depending upon the procedure being performed.

### TECHNICAL FIELD

This document relates to access to a body cavity, such as the peritoneum, any organ contained in the peritoneum, bladder, ureter, renal pelvis, or retroperitoneum.

### BACKGROUND

A number of medical procedures may require access to a targeted site in a body cavity. For example, some abdominal and pelvis procedures, including tubal ligation, appendectomy, gastrectomies, hysterectomies, colectomies, adrenalectomies, and the like, may use laparoscopic or endoscopic access. Laparoscopy may require small incisions made through the anterior abdominal wall. Via these incisions, a rigid laparoscope can be introduced into the body and toward the targeted site in the peritoneal cavity. An endoscope device may be used to access a targeted site in the peritoneal cavity by passing the distal end of the endoscope through an opening formed in the digestive tract (e.g., transgastric peritoneoscopy), such as an opening formed in the stomach wall. The endoscope device typically provides a single distal end having a lumen through which forceps, loops, or other instruments may be passed to treat or examine the targeted site.

Given the presence of a skin incision, laparoscopic access through the abdominal wall is associated with a risk of infection and development of postoperative hernias, scars, and adverse cosmetic results. While the transgastric approach with an endoscope device is performed without abdominal incisions and may reduce scars or adverse cosmetic results, the transgastric approach may be associated with chemical peritonitis, infection, and fistula formation. In addition, accessing the peritoneal space through the stomach wall can limit the ability to use rigid endoscopes.

### SUMMARY

Some embodiments of the system described herein provide transvesical access to a body cavity (e.g., the peritoneum, the bladder, the ureter, the renal pelvis, or the retroperitoneum). For example, such a transvesical approach may provide access to the peritoneal cavity through an opening formed in the bladder wall or other structures in the urinary tract (e.g., ureter, renal pelvis, or the like). Thus, the transvesical approach through the bladder wall permits a surgeon to examine, conduct surgical or therapeutic procedures, or a combination thereof inside peritoneal cavity. Further, the transvesical access to the peritoneal cavity or another body cavity provides the opportunity to use flexible or rigid endoscopes, depending upon the procedure being performed. In addition, the tranvesical access to the peritoneal cavity or another body cavity provides the opportunity to use robotic technology, including endoluminal robots, self contained miniaturized robots, or telerobotic platforms during the procedures being performed.

Particular embodiments of the intracavitary transvesical approach provide access to the peritoneal cavity, the retroperitoneal space (including the upper urinary tract), or both for the purpose of examination, therapeutic procedures, or selected surgical procedures. It should be understood after reviewing the description herein that the specific access technique and approach would depend on the locus of the targeted site. For example, the approach to the peritoneal space may be via the bladder or via another organ in the urinary tract, and the approach to the retroperitoneal space may be via the bladder or via another organ in the urinary tract.

Some or all of the embodiments described herein may provide one or more of the following advantages. First, the transvesical approach described herein may provide a direct line of site to many structures and organs in the peritoneal cavity due to the anatomical relationships of the bladder and peritoneal cavity. Second, the transvesical approach to the peritoneal cavity may separate the operating field away from the patient's airway (which may be constricted in a transgastric approach) and may provide separate working spaces in the operating room for the surgeon and the anesthesiologist and monitoring equipment. Third, the transvesical access technique can be performed using flexible or rigid endoscopes singularly or in multiplicity, unlike the typical transgastric approaches that are generally limited to flexible endoscopes. The ability to use rigid instrumentation is feasible given the anatomic relationships of the bladder to the peritoneal space (e.g., the urethra to the bladder to the peritoneal space may be substantially linear). Fourth, the transvesical approach may provide access to the targeted site in the peritoneal cavity without caustic gastric contents (e.g., substances in the stomach or intestines) necessarily spilling into the peritoneal space. Fifth, the transvesical access technique may be advantageously used for examination, therapy, or surgical procedures on the external surface of stomach, which may be difficult to access via a transgastric approach. Sixth, the bladder may be a sterile field so that communication of the bladder with the peritoneal cavity may not alone increase the risk of infectious complications. This is in contrast to transgastric or transcolonic peritoneoscopy in which nonsterile bowel contents may contact the peritoneal cavity and may increase the risk of intraperitoneal infection.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a transvesical access system being inserted into a urinary tract, in accordance with some embodiments.

FIG. 2 is a front view of an access sheath of the transvesical access system of FIG. 1 penetrating through a wall in the urinary tract and into the peritoneal cavity, in accordance with some embodiments.

FIG. 3 is a side view of an embodiment of the transvesical access system.

FIG. 4 is a side view of another embodiment of the transvesical access system.

FIGS. 5A-B are views of an access apparatus of a transvesical access system, in accordance with some embodiments.

FIGS. 6A-B are views of an access sheath of a transvesical access system, in accordance with some embodiments.

FIG. 7 is a side view of the access sheath of FIG. 6A and an optics system, in accordance with some embodiments.

FIG. 8 is a front view of an access sheath of FIG. 7 penetrating through a wall in the urinary tract, in accordance with some embodiments.

FIG. 9 is a side view of the access sheath of FIG. 6A and an instrument induction assembly, in accordance with some embodiments.

FIGS. 10A-F are views of the instrument induction assembly of FIG. 9.

FIG. 11 is a side view of the access sheath of FIG. 6A and an inner sheath device, in accordance with some embodiments.

FIGS. 12A-C are views of the inner sheath device of FIG. 11.

FIG. 13 is a perspective view of an inner sheath device in accordance with some embodiments.

FIGS. 14A-B are views of an inner sheath device in accordance with another embodiment.

FIG. 15 is a side view of a scaffolding system for releasably retaining components of a transvesical access system, in accordance with some embodiments.

FIGS. 16A-G are views of components of the scaffolding system in accordance with some embodiments.

FIG. 17 is an instrument holder for use with an access sheath, in accordance with some embodiments.

FIGS. 18A-C are views of an attachment portion of the instrument holder of FIG. 17 and a mating portion of an interchangeable device.

FIGS. 19A-B are views of an intracavitary needle driver device, in accordance with some embodiments.

FIGS. 20A-I are views of the intracavitary needle driver device of FIG. 19A, in accordance with some embodiments.

FIG. 21 is a side view of a distal portion of the intracavitary needle driver device of FIG. 19A.

FIGS. 22A-D are views of a three prong clip device, in accordance with some embodiments.

FIGS. 23A-B are views of needle viewing instruments, in accordance with some embodiments.

FIGS. 24A-C are views of an endoscope device and an endoscopic holster device, in accordance with some embodiments.

FIGS. 25 A-C are views of retraction devices for use in a body cavity, in accordance with some embodiments.

FIG. 26 is a section view of a retractor device used in the peritoneal cavity, in accordance with some embodiments.

FIG. 27 is a section view of a retractor device used in a laparoscopic procedure, in accordance with some embodiments.

Like reference symbols in the various drawings indicate like elements.

### DETAILED DESCRIPTION OF ILLUSTRATIVE EMBODIMENTS

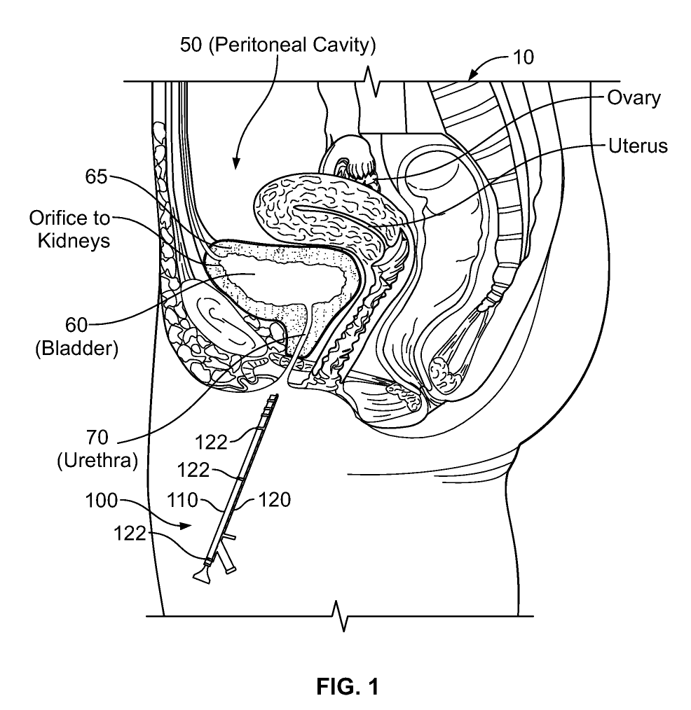

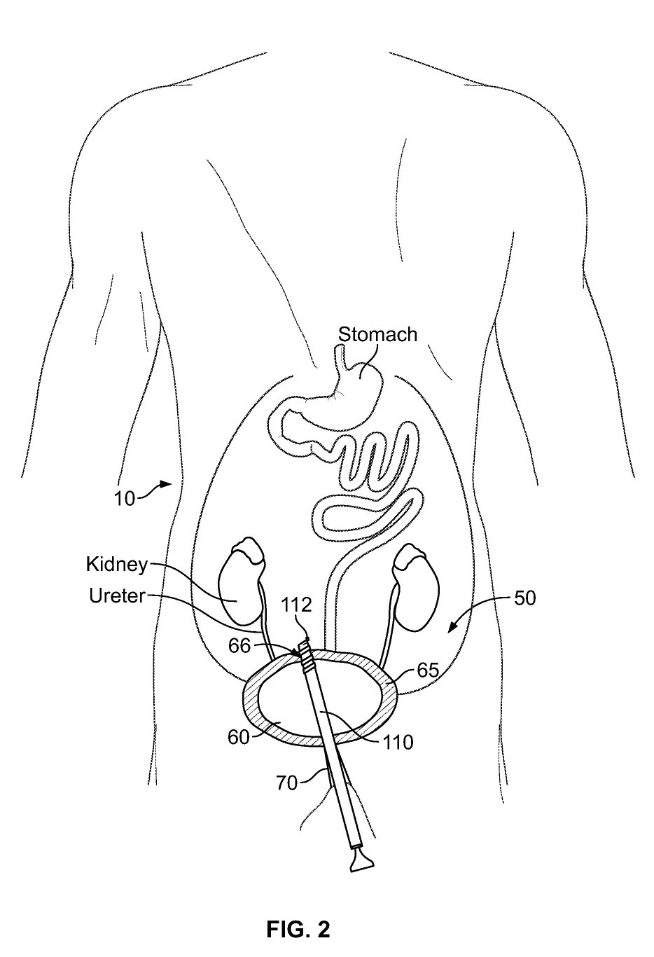

Referring to FIG. 1, some embodiments of a transvesical access system 100 may provide access to a body cavity through an opening formed in the bladder wall or other structures in the urinary tract (e.g., ureter, renal pelvis, or the like). For example, the transvesical system 100 may provide access to the peritoneal cavity 50 through an opening formed in the bladder wall 60 (described in more detail below in connection with FIG. 2). Such an intracavitary transvesical approach provides access to the peritoneal cavity 50 for the purpose of examination, therapeutic procedures, or selected surgical procedures, as described in more detail below.

The transvesical access system 100 may include an access sheath device 110 that is releasably coupled to an access apparatus 120. For example, the access apparatus 120 may include one or more sheath holder devices 122 are couple the elongate body of the access sheath to the elongate portion of the access apparatus 120. In such circumstances, the access apparatus 120 may facilitate the movement of the access sheath device 110 as they are passed through the urethra 70 and into the bladder 60. For example, the access apparatus may include an optics system (e.g., an endoscope device passing through an offset channel of the access apparatus) that provides direct vision of the urinary tract to the surgeon while the transvesical access system 100 approaches the bladder wall 65.

As shown in FIG. 1, the anatomic relationships of the bladder 60 to the peritoneal space 50 (e.g., the path to the urethra 70, to the bladder 60, and to the peritoneal space 50 may be substantially direct) can provide a transvesical approach to the peritoneal cavity 50. Such an approach may provide a generally direct line of access to many structures and organs in the peritoneal cavity 50. This direct line of access can, in some circumstances, permit the use of flexible and rigid endoscope devices. Also, the transvesical approach to the peritoneal cavity does not necessarily interfere with access to the patient's airway (which may be constricted in a transgastric approach), thereby separating working spaces in the operating room for the surgeon and for the anesthesiologist.

Referring now to FIG. 2, the transvesical system 100 may provide access to the peritoneal cavity 50 through an opening formed in a wall of the urinary tract, such as the bladder wall 65. In this embodiment, the access sheath device 110 is passed through the urethra 70 and into the bladder 60. As previously described, the access sheath device 110 may be inserted into the bladder 60 using the access apparatus 120 (FIG. 1) that is detachably coupled to the elongate body of the access sheath device 110. The access sheath device 110 may include an entry mechanism 112 (described in more detail below) that can penetrate the bladder wall 65 (or another wall in the urinary tract) to provide entry into the peritoneal cavity 50. In some embodiments, the access apparatus 120 remains in the bladder 60 while the access sheath device 110 penetrates through the bladder wall 65. As such, the optics system of the access apparatus 120 can provide direct vision of the bladder 60 during the penetration procedure. In addition, the access sheath 110 may receive a secondary optics system 130 (e.g., an endoscope device) that provides direct vision of the bladder wall 65 and the peritoneal cavity 50 during the penetration procedure (described in more detail below). For example, at least the distal portion of the access sheath 110 may comprise a substantially transparent or translucent material so that the secondary optics system 130 disposed in the instrument channel of the sheath 110 can provide direct vision of the bladder wall 65 (e.g., permits the surgeon to monitor for excessive bleeding and to monitor for entry into the peritoneal cavity 50. As the distal portion of the access sheath device 110 penetrates into the peritoneal cavity 50, the instrument channel of the access sheath device 110 enters into communication with the peritoneal cavity 50 to provide access thereto. Such a transvesical approach provides access to the peritoneal cavity 50 for the purpose of examination, therapeutic procedures, or selected surgical procedures, as described in more detail below.

The embodiment depicted in FIG. 2 shows a transvesical approach to the peritoneal cavity 50 by way of example. It should be understood after reviewing the description herein, however, that such transvesical techniques can be applied to provide access to other body cavities or spaces, including retroperitoneal access from the bladder or other structures of the urinary tract.

In preparation for using the transvesical access system 100 to access, examine, and perform surgical procedures or therapy in the peritoneal space, the external genitalia of the patient 10 and lower abdominal region may be sterile prepared and the instrumentation of the transvesical access system 100 may be sterilized. As previously described, the anatomical relationship of the bladder 60 and peritoneal cavity 50 may permit a direct line of approach to the targeted structures in the peritoneal cavity 50. Such a direct line of approach permits the transvesical access system 100 to employ flexible or rigid endoscope devices singularly or in multiplicity (rather than being limited to only one type of endoscope device). In some circumstances, the maximum diameter of the urethra 70 may be larger than the diameter of certain laparoscopic instruments, so the transvesical access system 100 may employ a rigid laparoscopic instrument in addition to or as an alternative to rigid endoscope devices. In addition, the patient 10 may be positioned on the operating table in a manner to enhance the direct line of approach to the peritoneal cavity. For example, in some circumstances, use of Trendelenburg and lateral tilt positioning will facilitate access to targeted organs in the peritoneal cavity.

Still referring to FIG. 2, the transvesical access system 100 may be employed to approach the peritoneal cavity 50 or another body cavity without constricting the airway of the patient 10. For example, the transvesical access system 100 may be implemented without instruments passing through the patient's mouth, throat, and esophagus, which may be constricted during a conventional transgastric approach. Accordingly, the use of a transvesical approach to the peritoneal cavity 50 may move the surgical field away from the patient's airway and provides an operating room environment in which the surgical field is separate from the anesthesiologist and monitoring equipment.

In some embodiments, during use of the transvesical access system 100, conduits for passage of endoscopes and other instrumentation may isolate remaining portions of the urinary tract from the access performed in the intraperitoneal space. Such isolation may reduce or prevent distension of the remaining portions of the bladder with fluid and may facilitate ease of instrument introduction to the peritoneal space. The normal anatomy of the ureterovesical junction provides for a non-refluxing mechanism thereby eliminating concern for reflux of fluid into structures of the upper urinary tract (e.g., ureter, renal pelvis, and kidney). In general, the urinary tract is separate from the gastrointestinal tract, so insufflation of fluid through the urinary tract will not result in potential gaseous distension of the bowels that could result in impaired access to the target structure in the peritoneal space. Furthermore, caustic gastric contents in the gastrointestinal tract will not spill into the peritoneal space when using particular embodiments of the transvesical approach. It should be understood, that in some circumstances, extravasation of sterile urine into the peritoneal cavity 50 might occur during the transvesical approach through the bladder wall, but this phenomenon has occurred during other laparoscopic and open urologic interventions in the peritoneal space without sequelae or serious side effects.

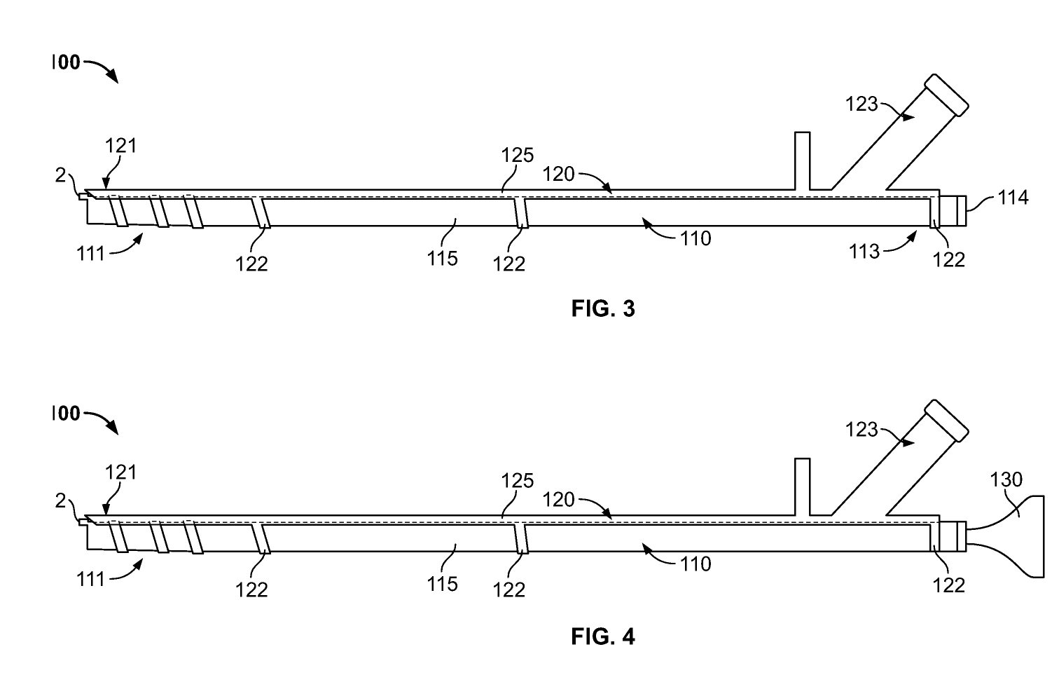

Referring now to FIG. 3, the transvesical access system 100 includes the access sheath device 100 having an elongate body 115 that is configured to be releasably coupled to the access apparatus 120. In this embodiment, the access apparatus 1210 includes a plurality of sheath holder devices 122 (e.g., thin metallic bands or the like) that releasably engage the elongate body 115. The sheath holder devices 122 may extend from a main body 125 of the access apparatus 120 to form a whole or partial loop that engages the access sheath device 110. In this embodiment, the sheath holder devices 122 curve to have an inner diameter of about 5 mm to about 15 mm, about 6 mm to about 12 mm, and preferably about 10 mm. Such a size may correspond to the outer diameter size of the elongate body 115, which can be, for example, about 5 mm to about 15 mm, about 6 mm to about 12 mm, and preferably about 10 mm. In some embodiments, the elongate body 115 may include one or more annular grooves formed therein to receive the sheath holder devices 122 of the access apparatus 120. The access sheath device may be provided in one or more lengths to accommodate differences in urethral length among men and women. For example, the access sheath device in this embodiment is capable of traversing the entire length of the urethra, bladder, and bladder wall for use in either men or women.

The access sheath device 110 may be coupled to the access apparatus 120 to facilitate insertion of the distal portion 111 of the access sheath device 110 through the urethra 70 and into the bladder 60 (FIG. 1). For example, the access apparatus 123 may include an optic system 123 (e.g., an endoscope device or the like) that extends through the main body 125 toward the distal end 121. In such circumstances, the access apparatus 120 may provide direct vision to the surgeon of the urinary tract as the access sheath device 110 is directed toward the bladder wall 65 (or another wall of the urinary tract). It should be understood, however, that the access sheath device 110 may be inserted into the bladder 60 without employing the access apparatus 120. The access apparatus 120 may be optionally employed to increase the safety of transvesical access due to the contemporaneous monitoring of the bladder 60 as the access sheath device 110 penetrates through the bladder wall 65. In such circumstances, after the access sheath device 110 has penetrated into the peritoneal cavity 50, the access apparatus 120 can be released from the access sheath device 110 and removed from the patient 10.

Referring to FIG. 4, in some circumstances, the access sheath device 110 may receive a second optics system 130 in its instrument channel so that the surgeon can monitor the bladder wall 65 and the peritoneal cavity 50 during penetration into the peritoneal cavity. This optics system 130 may extend through the instrument channel toward the distal portion 111 to provide direct vision of the tissue or area near the distal portion 111 of the access sheath device 110. Accordingly, the transvesical access system 100 may provide contemporaneous monitoring (e.g., using the first optic system 123 of the access apparatus 120 and using the second optics system 130 received in the access sheath device 110) during advancement into the peritoneal cavity 50. In those embodiments in which the access sheath device 110 is inserted into the bladder 60 without the use of the access apparatus, the second optic system 130 may provide direct vision of the urinary tract to the surgeon.

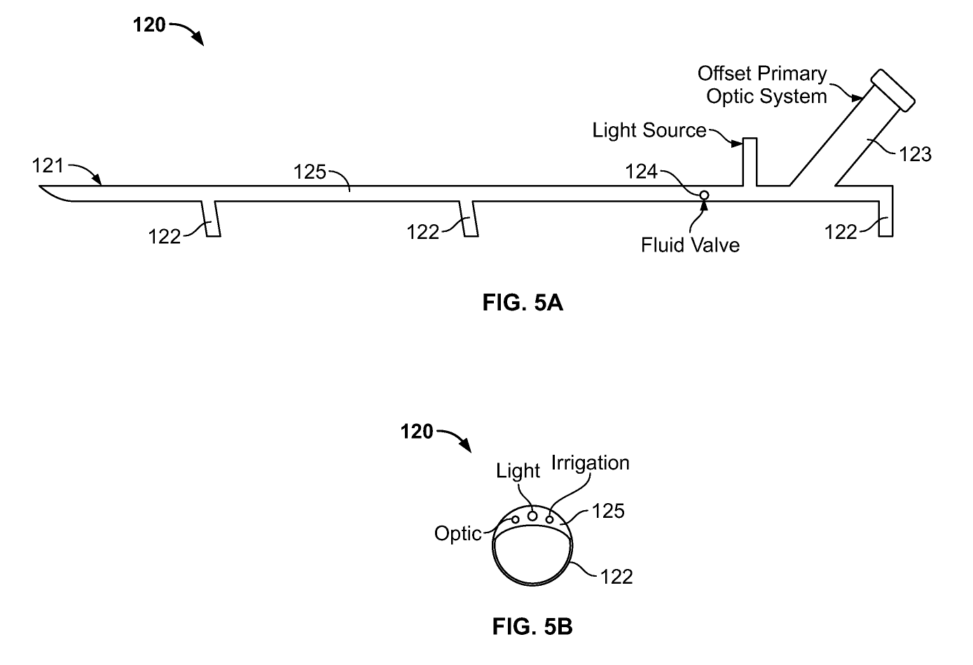

Referring to FIGS. 5A-B, the access apparatus 120 may include a plurality of channels (e.g., optic, light, irrigation, or the like) extending longitudinally toward the distal portion. In this embodiment, at least a portion of the first optics system 123 may extend through the optics channel toward the distal portion 121 to provide direct vision of the urinary tract during insertion. Also, the access apparatus may include a light source port in communication with the light channel so that the space near the distal portion 121 may be illuminated (for use by the optics system). Also, in some embodiments, the access apparatus 120 may include a fluid valve 124 that is in fluid communication with the irrigation channel. For example, the fluid valve 124 and irrigation channel may be used to overfill the bladder with sterile saline or sterile glycine before the access sheath device engages the bladder wall 65 (FIG. 2).

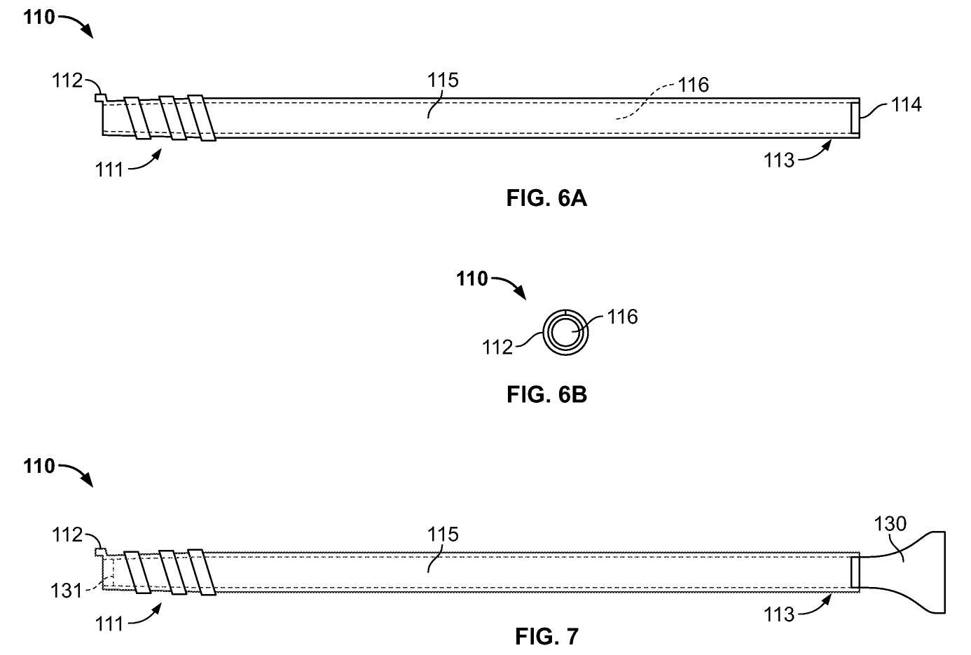

Referring to FIGS. 6A-B, the access sheath device 110 may include an entry mechanism 112 disposed along a distal portion 111. In this embodiments, the entry mechanism is a blundt entry screw mechanism that engages the bladder wall 65 (or another wall of the urinary tract) without requiring a sharp needle-like tip. The blundt entry screw mechanism 112 may include one or more tissue engagement threads that extend along the distal portion and that engage the bladder wall 65 after penetrating into the peritoneal cavity 50 (FIG. 2). In other embodiments, alternative blunt entry mechanisms (e.g., radial balloon dilation) or non-sharps cutting insertion assemblies (e.g., blunt winged designs) could be utilized to penetrate a wall or the urinary tract. The access sheath device 110 may also include at least one instrument channel 116 extending from an opening 114 at a proximal portion 113 and out through the distal portion 111. In these embodiments, the absence of sharps (e.g., blades, needles, scalpels, or the like) during penetration through the bladder wall 65 can minimize risk of access related complications. As previously described, the instrument channel 116 may receive an optics system (e.g., an endoscope device or the like) or other instruments employ during the transvesical access or for the purpose of examination, therapeutic procedures, or selected surgical procedures in the peritoneal cavity 50. In some circumstances, a needle penetration device or other sharp device could be inserted through the instrument channel 116 to initially puncture or otherwise engage the bladder wall 65 before the entry mechanism 112 is employed.

Referring to FIG. 7, the access sheath device 110 may receive an optics system 130 in the instrument channel 116, as previously described. The optics system 130 may include an image capturing device 131 disposed at its distal end. In some circumstances. The image capturing device 131 may be disposed in the instrument channel 116 in the distal portion 111 of the access sheath device 110. As such, image capturing device 131 may record video or other provide direct vision of the bladder wall 65 and the peritoneal cavity 50 during the penetration procedure.

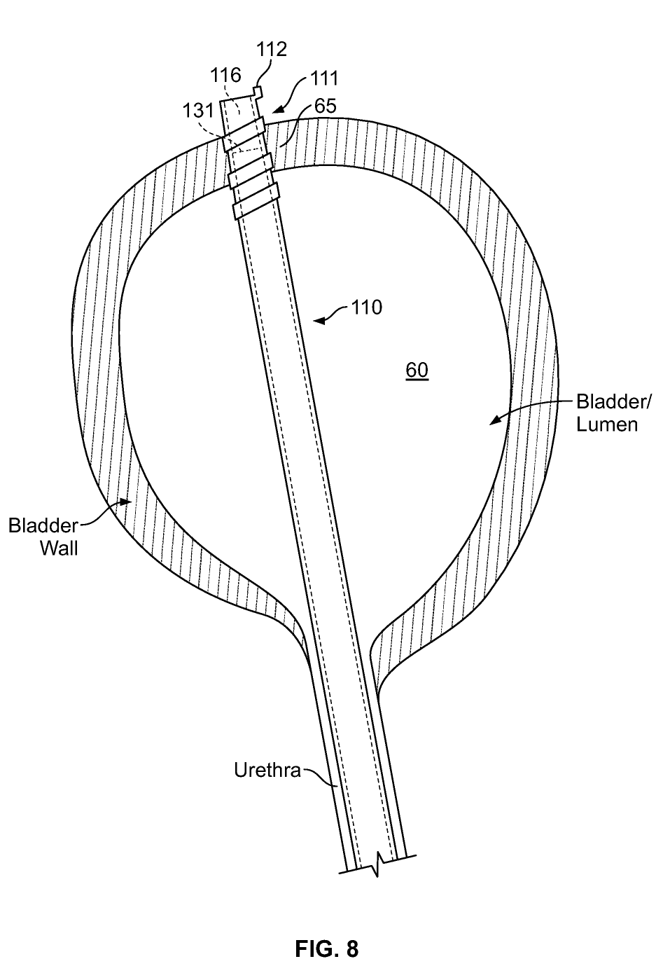

Referring to FIG. 8, at least the distal portion 111 of the access sheath device 110 may comprise a substantially transparent or translucent material so that the image capturing device 131 may provide direct vision of the bladder wall 65 that has been engaged by the entry mechanism 112. After the distal portion 11 of the access sheath device 110 has penetrated into the peritoneal cavity, the optics system 130 may be removed from the instrument channel 116. Alternative, the optics system 130 may remain in a first instrument channel while other instruments are passed through a second instrument channel (not shown in FIG. 8) passing through the access sheath device 110.

In one example of placing the access sheath device 110 through the bladder wall 65, the access sheath device 110 may be releasably engaged by the sheath holder devices 122 of the access apparatus 120 (shown, for example, in FIG. 3). Under direct endoscopic vision using the first optic system 123, the access apparatus 120 (and the access sheath device 110 connected thereto) can be placed into the bladder 60. Then, sterile saline or sterile glycine can be used to overfill the bladder 60 using the irrigation channel of the access apparatus 120 (shown, for example, in FIG. 5A-B). After the penetration site on the bladder wall 65 has been selected, the access sheath device 110 may engage the bladder wall 65 under contemporaneous monitoring of the first optics system 123 and the second optics system 130. After the access sheath device 110 has penetrated through the bladder wall 65 and into the peritoneal space, the access apparatus 120 may be released from the sheath device 110 and remove fro the patient 10 while the access sheath device 110 remains in communication with the peritoneal space (as shown, for example, in FIG. 8).

Still referring to FIG. 8, the access sheath device 110 can comprise a nonconductive material, such as a biocompatible polymer material. As previously described, at least a portion of the access sheath device 110 may comprise a substantially transparent material to facilitate monitoring of the bladder wall 65 during penetration. A blunt entry screw mechanism will be incorporated in the design. In other embodiments, the access sheath device 110 may comprise a metallic material, such as stainless steel.

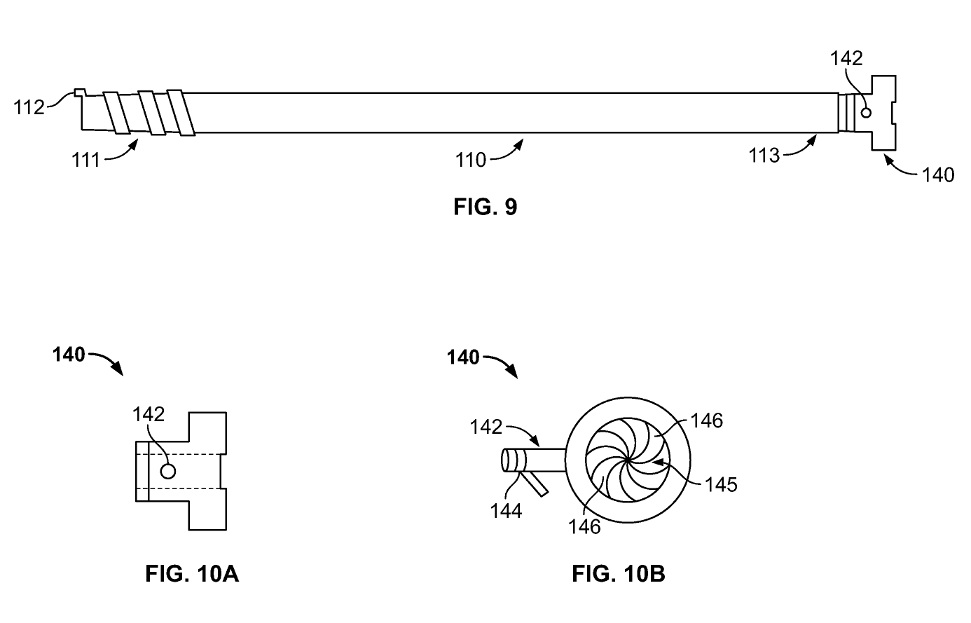

Referring now to FIG. 9, an instrument induction assembly 140 may be coupled to the proximal portion 113 of the access sheath device 110. The instrument induction assembly 140 may include a detachable locking assembly that can be fixed to the body of the access sheath device 110. For example, the instrument induction assembly 140 may be connected to the access sheath device 110 after the sheath device 110 has penetrated into the peritoneal cavity. As described in more detail below, the instrument induction assembly 140 may be used provide a substantial seal at the proximal portion 113 of the access sheath device 110.

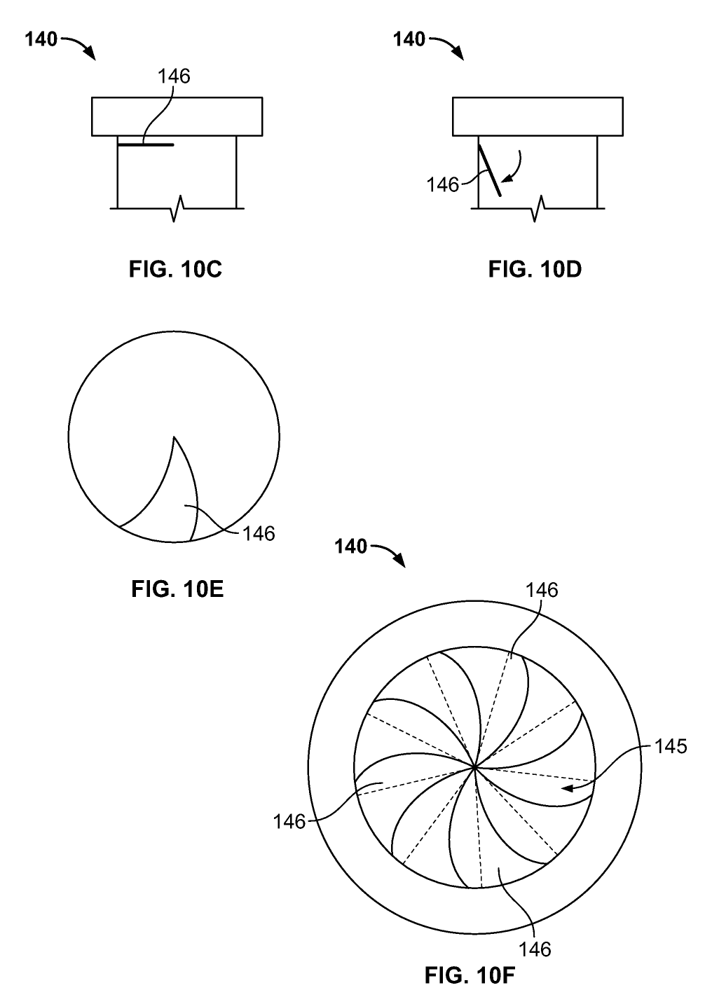

Referring to FIGS. 10A-F, the instrument induction assembly 140 may include a fluid insufflation (or instillation) assembly 142. For example, the fluid insufflation assembly may include a valve 144 that permits the passage of carbon dioxide or another fluid through the access sheath device 110 and into the peritoneal cavity 50. In some circumstances, the peritoneal cavity 50 may be insufflated with carbon dioxide or another fluid to create a working space inside the peritoneal cavity 50. It should be understood that, in some embodiments, the instrument induction assembly 140 may include a fluid instillation assembly to deliver a liquid rather than a gaseous fluid.

As shown in FIGS. 10B-F, the instrument induction assembly may include a plurality of leaflets 146 that provide a swirl pattern 145 at the proximal opening. The leaflets 146 may be arranged in the swirl pattern 145 to provide a seal at the proximal opening of the instrument induction assembly 140, thereby substantially preventing the carbon dioxide or other fluid from seeping out of the peritoneal cavity. When an instrument is passed through the instrument induction assembly 140 (e.g., through the access sheath device 110 and into the peritoneal cavity), at least a portion of the leaflets 146 may be shifted distally toward the access sheath device. The leaflets 146 may comprise a polymer material or another flexible material so that the leaflets are generally biased against the instrument that is inserted into the access sheath device 110. Such a biasing effect provides at least a partially seal along the proximal opening o the instrument induction assembly 140.

Accordingly, some methods of transvesical access to the peritoneal cavity may include reliably connection the access sheath device to the access apparatus 120 (FIG. 1). As previously described, in some embodiments, the access sheath device 110 can slide into the sheath holder devices 122 (e.g., metallic bands) that frictionally hold the access sheath device 110 to the main body 125 of the access apparatus 120. Then the access apparatus 120 (with the access sheath 110 coupled thereto) may be inserted through the urethra 70 and into the bladder 60 under direct vision. In some circumstances, the bladder 60 may be overfilled with saline (e.g., using the irrigation channel of the access apparatus 120). After the penetration site in the bladder wall 65 is identified, the second optics system 130 may be inserted into in the instrument channel 116 of the access sheath device 110. Using direct vision of the bladder 60(via the offset optics system 123 of the access apparatus 120) and using direct vision of the bladder wall 65 (via the second optics system 130 received in the access sheath device 110), the distal portion 111 of the access sheath device 110 may penetrate through the bladder wall 65 and into the peritoneal cavity 50, thereby providing transvesical access. After transvesical access is provided, the access sheath device 110 can be anchored into the bladder wall 65 using the threads of the entry mechanism 112. The second optics system 130 may then be removed from the access sheath device 110. Also, the access apparatus 120 may be removed from the access sheath device 110 and removed from the patient's body 10. (As the access apparatus 120 is removed from the access sheath device 110, the surgeon may hold the access sheath device 110 in position.) At this point in the procedure, the access sheath device 110 may be the only piece of instrumentation in the body 10 (FIG. 8). Then the instrument induction assembly 140 may be attached to the proximal portion 113 of the access sheath device 110 so that insufflation of carbon dioxide can be performed. Thereafter, other instruments described herein may be passed through the access sheath device 110 for the purpose of examination, therapeutic procedures, or selected surgical procedures in the peritoneal space.

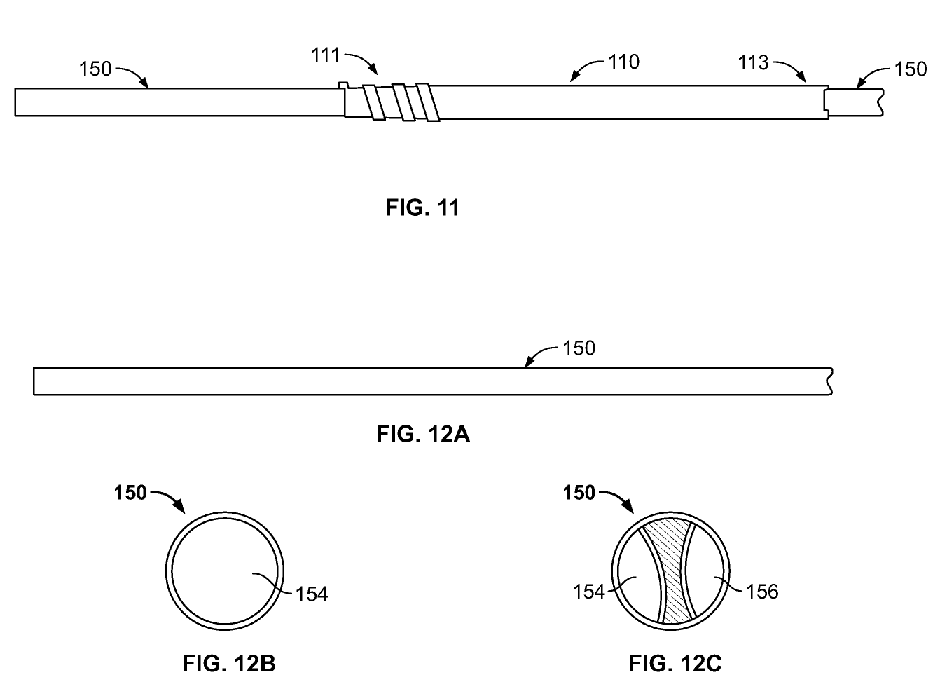

Referring now to FIG. 11, one or more inner sheath devices 150 may be advanced through the access sheath device 110 and into the peritoneal cavity. The inner sheath device 150 may be specially configured for a unifunctional purpose or may be employed in multiple functions. The inner sheath devices 150 may provide the opportunity for examination, therapeutic procedures, or surgical capabilities to targeted tissues in the peritoneal cavity 50 (FIG. 1). In some embodiments, the inner sheath device 150 may be attachable to the access sheath device 110, for example, using a detachable locking device (e.g., a threaded engagement or the like) at the proximal portion 113 of the access sheath device 150. The configuration of the inner sheath device 150 may be tailored to the needs of the intervention being performed transvesically in the peritoneal cavity 50. For example, the inner sheath device 150 may comprise a nonconductive polymer material and may be provided in a variety of shapes, lengths, and/or sizes to permit the intraperitoneal intervention. In some embodiments, the inner sheath device 150 can made from a metallic material, for example, to assist with examination, therapy, and surgical procedures in the peritoneal space. In other embodiments, the inner sheath concept can be used in the lumen of other body cavities or organs. For instance, an inner sheath may be useful in navigating an endoscope through a tight bend or angulation of the large or small bowel. In other embodiments, the inner sheath concept may be useful for access to select calices in the renal collecting system during flexible ureteroscopy.

The inner sheath device may be configured to enhance a number of aspects of intracavitary interventions and specifically transvesical intraperitoneal interventions including diagnostic, therapeutic, or surgical interventions. In some embodiments, the inner sheath device 150 is designed to enhance operating in the transvesical intraperitoneal operative field by providing additional stability at the operative field. For example, if the operative field includes the gall bladder, the inner sheath device 150 may enhance or augment endoscopic evaluation, treatment or surgical intervention specifically at the target location of the gall bladder.

Referring to FIGS. 12A-B, the inner sheath device 150 may include a number of different channel configurations. For example, as shown in FIG. 12 B, the inner sheath device 150 may have a single channel 154 through which one or more instrument may pass during inventions in the peritoneal cavity. In another example, as shown in FIG. 12C, the inner sheath device 150 may have multiple channels 154 and 165. Depending on the planned function of the transvesical intraperitoneal intervention, a set of specialized inner sheath devices 150 can be employed throughout the procedure.

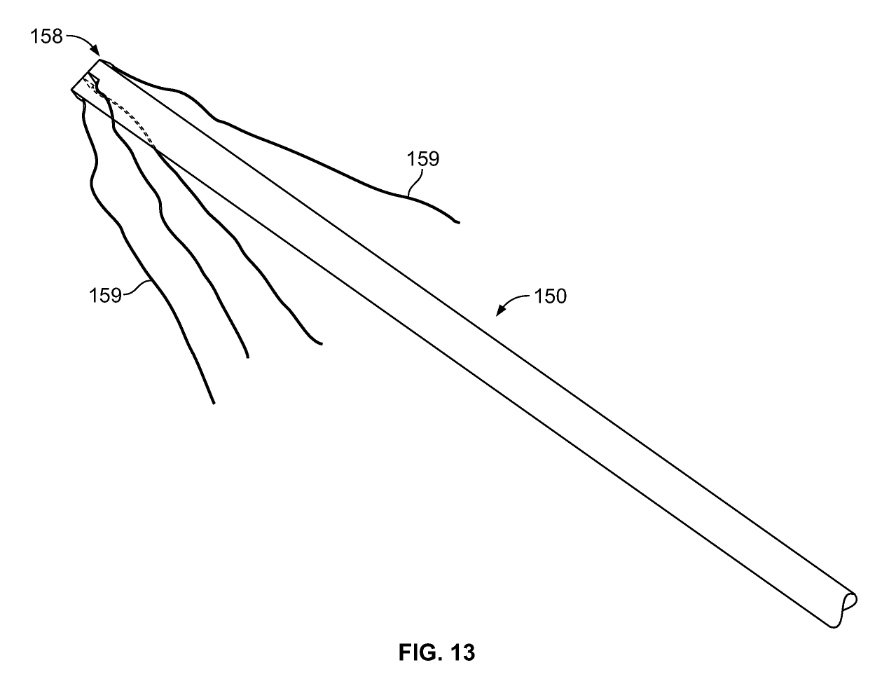

Referring to FIG. 13, the inner sheath device 150 may comprise a flexible distal portion that permits steerability. For example, a flexible steerable inner sheath device 150 may incorporate a steering mechanism 158. The steering mechanism 158 may comprise anchoring mechanisms from which a set of steering lines 159 extend. The steering lines 159 may extend to a distance at least double the length of the actual inner sheath device 150. In some embodiments, the steering lines 159 may comprise silk suture, one or more polymers, metallic wires, or the like. Also, the steering lines 159 may comprise tubes comprised of metal or polymers, rods, ribbons, and/or bars comprised of metal or polymers will also be considered as steering and anchoring mechanisms. As shown in FIG. 13, the steering lines 159 may extend along the outside of the inner sheath device 150. The steering lines 159 can be attached to a proximal anchoring mechanism located, for example, on the instrument induction assembly 140 attached to the access sheath device 110. Although flexible inner sheath devices 150 are described herein, it should be understood, however, that some embodiments of the inner sheath devices may comprise a substantially rigid material, including some polymer and metal materials.

In use, the inner sheath steering mechanism 158 may be used to direct the distal end of the inner sheath device 150 to a targeted tissue site. For example, the steerable inner sheath device 150 (FIG. 13) may be positioned through the access sheath device 110 into the peritoneal cavity 50. If an adjustment is needed in the leftward direction to reach the targeted tissue, the left-sided steering lines 159 would be pulled resulting in leftward deflection of the steerable inner sheath device 150. Tension on the left-sided steering lines 159 can be maintained by fixing the left-sided steering lines 159 to the proximal anchoring mechanism located, for example, on the instrument induction assembly 140. If an adjustment is then needed in the rightward direction to reach the targeted tissue, then the left-sided steering lines could be released and the right-sided steering lines 159 may be grasped, pulled and stabilized resulting in a new leftward deflection on the steerable inner sheath device 150.

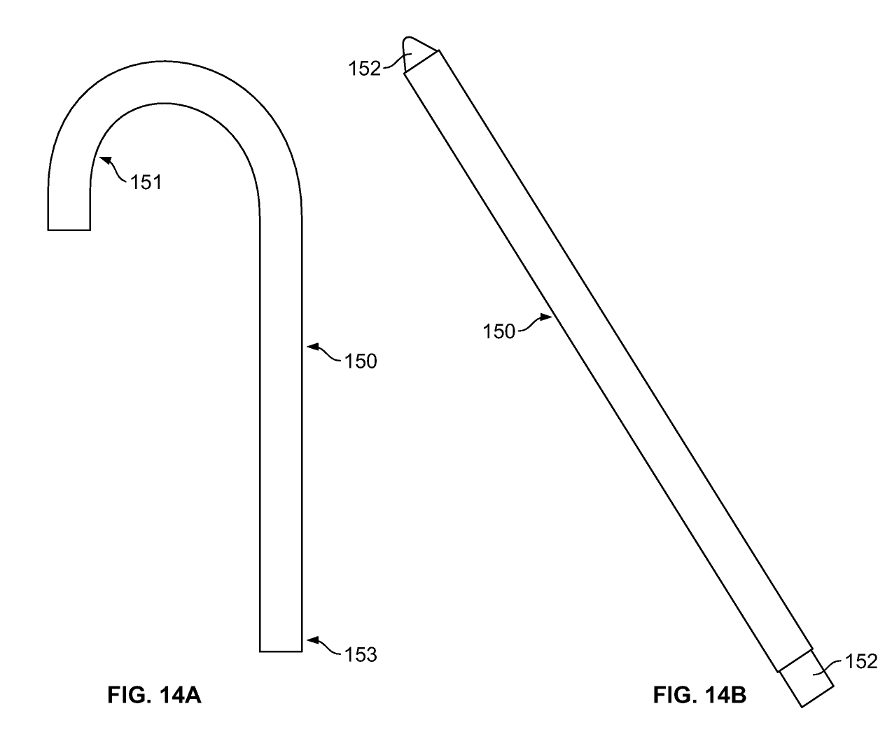

Referring to FIGS. 14A-B, in some embodiments, at least a portion of the inner sheath device 150 may comprise a shape-adjusting material, such as a memory material or a malleable material. For example, the distal portion 151 of the memory material inner sheath device 150 may have a specific configuration that could include varying inner sheath diameters, bends, angles, inner sheath swirls, or other shape arrangements (see, for example, FIG. 14A). As shown in FIG. 14B, such a memory material inner sheath device 150 can be straightened into a linear arrangement with the use of a substantially rigid guidewire 152 (or purpose-built obturator introducer assembly device). The straightened memory material inner sheath device 150 could then be introduced into the peritoneal cavity through the access sheath device 110. After being positioned inside the peritoneal cavity 50, the guidewire 152 may be removed from the inner sheath device 150 so that the inner sheath device can be restored to the memorized shape (see, for example FIG. 14A) that facilitates the intervention inside the peritoneal space.

In another alternative embodiment, a shape-adjusting inner sheath device 150 may comprise a malleable material that can be reshaped after being inserted into the peritoneal cavity. For example, the malleable inner sheath device may be composed of a specialized polymer or other substance that could be molded intraperitoneally with pressure of the inner sheath device on the lateral or anterior abdominal wall. After being molded into the desired configuration, the inner sheath device 150 may then be used for placement of an endoscope and/or instrumentation within the peritoneum to the targeted location in the surgical field.

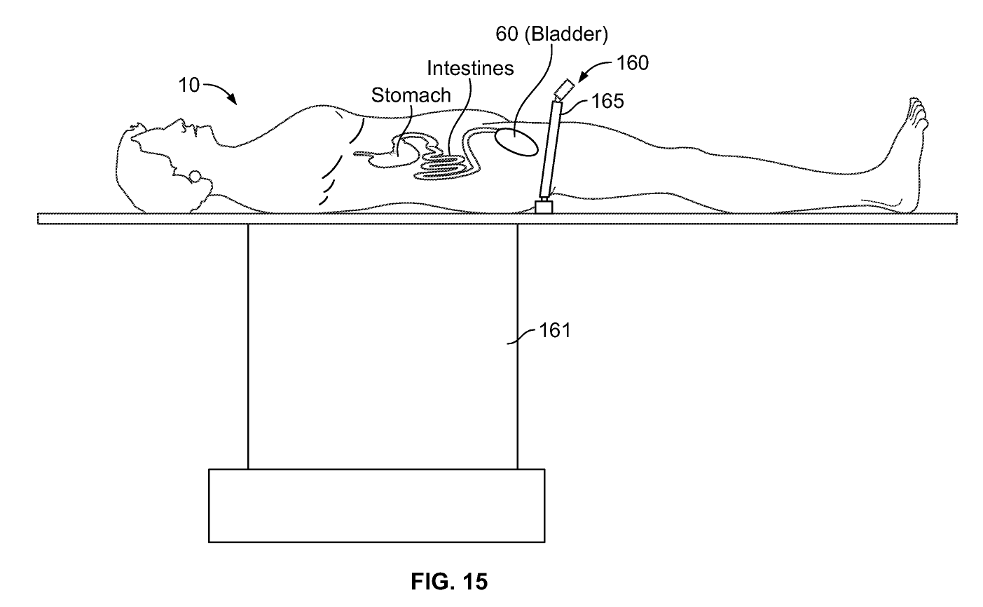

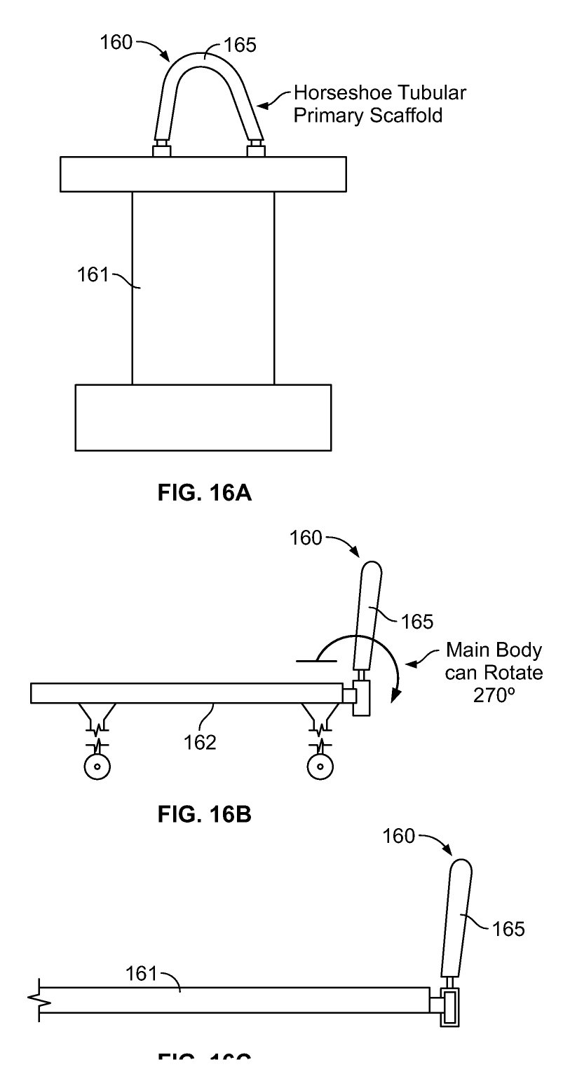

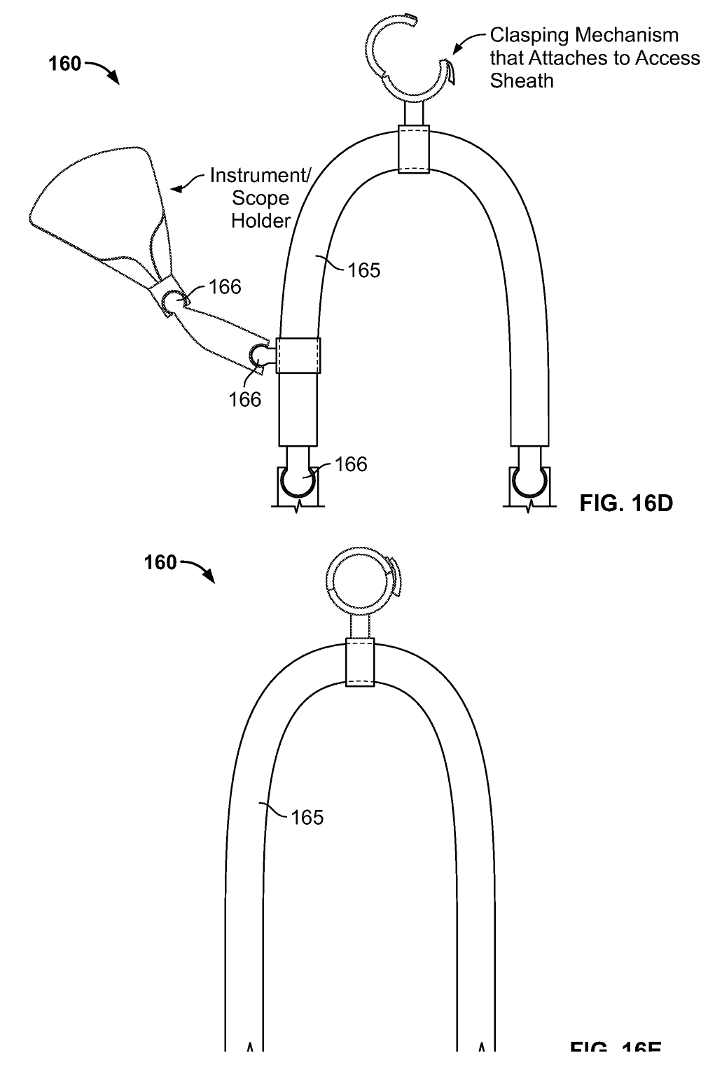

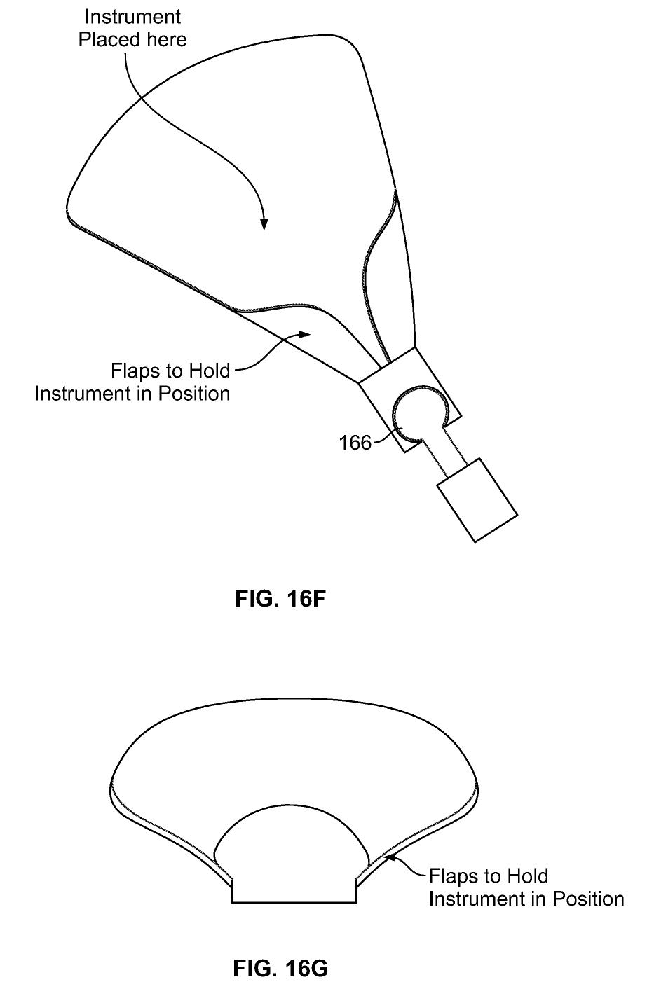

Referring to FIG. 15 and FIGS. 16A-G, some embodiments of the transvesical access system 100 may include an external access instrumentation scaffolding system 160. For example, one or more endoscopes may be used simultaneously during a transvesical intraperitoneal intervention, and the scaffolding system 160 may be employed to facilitate such tasks using one or more endoscope devices. The external access instrumentation scaffolding system 160 may be mounted to the patient's operating room table 161. Alternatively, the scaffold system 160 can be attached to a long nonconductive skid that is pushed under the patient after the patient is under anesthesia. The scaffolding system 160 can provide an ergonomic setup for the operating surgeon to perform transvesical intraperitoneal surgery. The scaffolding system 160 may have multiple holders or other attachment mechanisms for endoscopes concurrently being used as part of the intervention.

In some embodiments, the main body of the scaffold system 160 would be a horseshoe-shape scaffold 165 comprising a metal tubing of approximately 2 cm diameter. A ball and socket joint would be present near the attachment point to the bed such that the scaffold 165 could be rotated 270-degrees relative to the horizontal of the bed. To the metal tubing, a variety of vice grip type instruments could be attached. There would be in addition a variety of end effectors for the attachments including an access sheath holder (that would have a clasp holder design) and instrument holders. Ball and socket joints 166 could also be used for the attachments to increase the functionality of the design. Such attachments may be connected for a variety of instrumentation and equipment used in the description of the invention. For example, the access sheath device 110 can be attach to the external scaffolding system 160 after the access sheath device 110 has penetrated into the peritoneal cavity 50. When multiple endoscopes are in place during transvesical peritoneal interventions, the scaffolding system 160 may permit one endoscope to be held in an endoscope holder while using the other endoscope. In some embodiments, the scaffolding system 160 can also be used to support multiple endoscopes simultaneously thereby given the surgeon more control over all endoscopes involved in the intervention. In situations where both endoscopes are being used simultaneously, the scaffolding system 160 can retain other components (instruments, endoscopes, equipment) used during the transvesical peritoneal interventions described herein. Although described for transvesical peritoneal interventions, it should be understood that the external scaffolding system 160 can be employed for other endoscopic procedures involving all organ systems in the body. Furthermore, it should be understood from the description herein that the external scaffolding system 160 may also be employed for other diagnostic or surgical procedures performed in open fashion via the vagina or via an incision made in the perineum.

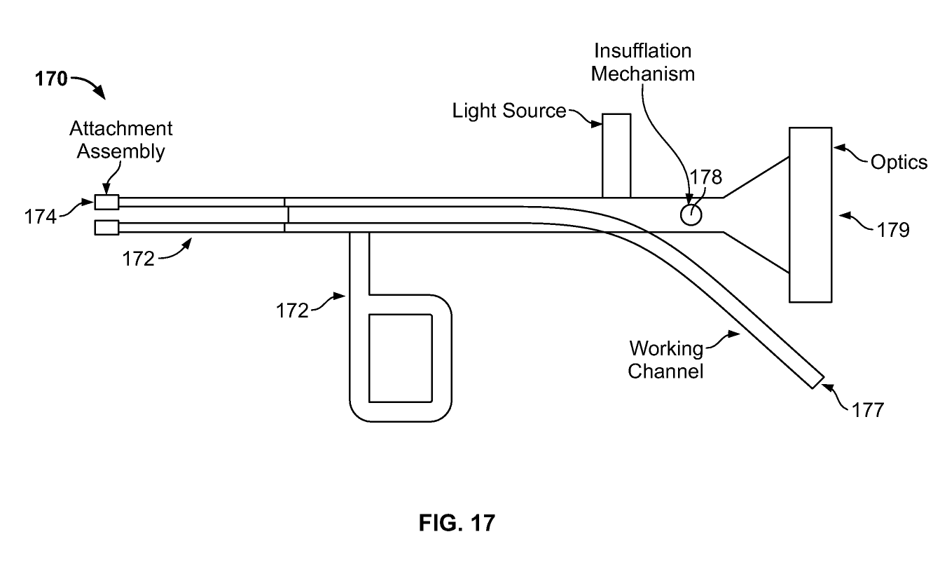

Referring to FIG. 17, some embodiments of the transvesical access system 100 may include an interchangeable instrument holder device 170. The interchangeable instrument holder device 170 may be used to hold a variety of instruments also described herein. In some embodiments, the instrument holder 170 is a reusable device that includes a variety of components, such as an instrument motion system 172 with four degrees of freedom plus grip, attachment assembly 174 for insertion of the interchangeable instruments, handle for instrument gripping, accessory working port 177, insufflation mechanism 178, an optics system 179, or a combination thereof. In some examples, the interchangeable instrument holder 170 may releasably attach to a variety of instruments configured to pass through the access sheath device 110, such as a scalpel, scissors, suturing device, needle driver, suture pushing device, biopsy forceps, stapling device, clip applier, specimen retrieval device, specimen morcellation device, and intracavitary injector/applicator/hollow needle/suction probe assembly. Through the working port 177, the instrument holder device 170 may also facilitate introduction of a various ablation system, laser fibers, probes, guidewires, instruments, additional optic systems, and radiographic imaging systems.

In some embodiments, the instrument holder 170 may be placed through the access sheath device 110 (or through an inner sheath device 150 disposed in the access sheath device 110) to facilitate performance of interventions in the peritoneal space. As such, the instrument holder 170 may be designed for use in concert with the access sheath device 110 when performing transvesical peritoneal procedures. In this embodiment, the instrument holder 170 may attach to the instrument induction assembly 140 (FIG. 9) attached to the access sheath device 110. It should be understood that such an instrument holder 170 can be used in conjunction with standard transurethral surgery. In these circumstances, the instrument holder 170 may attach to the instrument sheath for a cystoscope or resectoscope. As such, the instrument holder 170 may be configured to fit a cystoscope/resectoscope sheath as well as the transvesical access sheath device 110. Furthermore, it should be understood from the description herein that such an instrument holder 170 may also be used in conjunction with laparoscopic procedures.

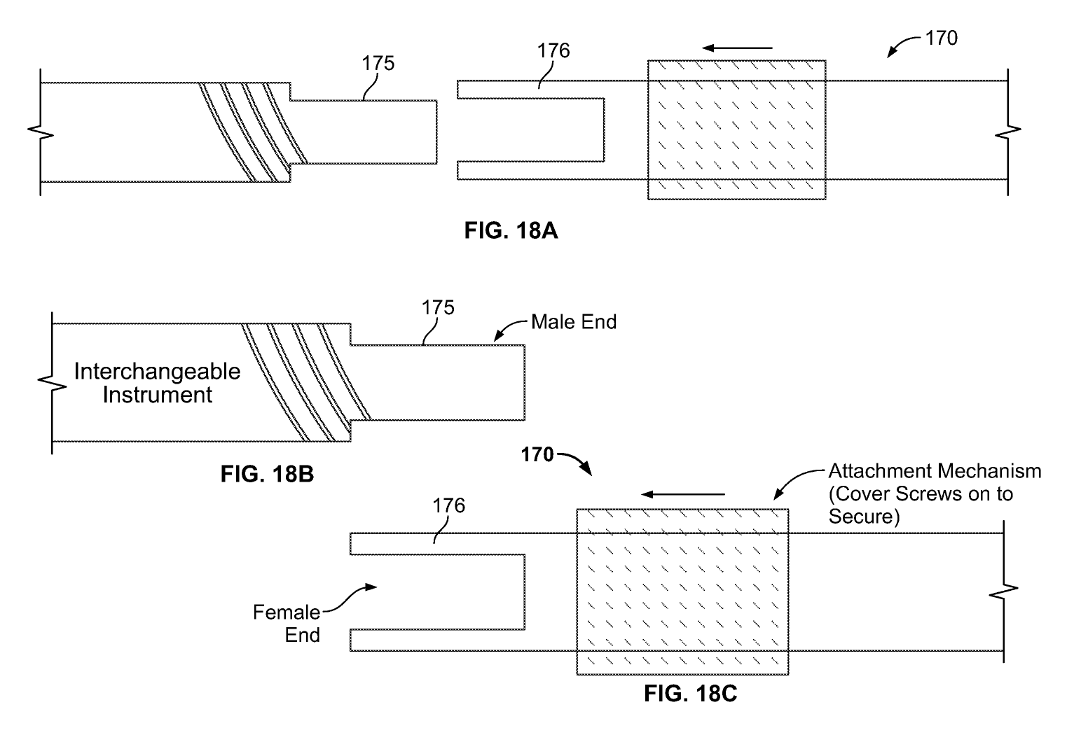

Referring to FIGS. 18A-C, the attachment assembly 174 may provide a connection point for the instruments to attach to the instrument holder 170. In some embodiments, the interchangeable instruments may include a male connector 175 that can be attached directly to a mating female connector 176. In some circumstances, a threaded engagement may be employed to lock the male connector 175 into the female connector 176. For example, if a needle driver instrument is to be inserted into the peritoneal cavity, the needle driver instrument can be attached to the instrument holder 170 outside of the patient's body 10. Then the instrument holder 170 (and the needle driver instrument connected thereto) can be advanced via the access sheath toward the peritoneal cavity. In other embodiments, some of the proposed instruments may include a combination of an instrument component attached to the attachment assembly 174 and another component passed through the working channel 177. For example, some instruments used in endoscopic surgery may utilize the working channel 177. In another example, if the access sheath device were to dislodge from the bladder wall 65, a guidewire could be placed through the working channel 177 into the peritoneal cavity to aid the process of regaining access.

For some interventions performed using the transvesical intraperitoneal approach, an intracavitary scalpel device may be utilized. Some embodiments of the scalpel device may be used for inventions directly in the peritoneal cavity or to expand the incision in the bladder during the intraperitoneal intervention. The scalpel device can be used in conjunction with the interchangeable instrument holder 170 (FIG. 17). The scalpel system may have two jaws that could engage tissue targeted for division. This design can provide an added element of safety in that only tissue within the jaws of the grasping mechanism could be cut. After assuring that tissue engaged in the grasping mechanism is safe for cutting, the cutting blade would be deployed using a blade pushing mechanism. The cut tissue may then be released.

In some embodiments, intracavitary scissors may be utilized in the peritoneal space. The scissors can be attached to the interchangeable instrument holder device 170 (FIG. 17) and passed through the access sheath device 110.

In some embodiments, an intracavitary suturing device can be utilized in the peritoneal space. The suturing device can be attached to the interchangeable instrument holder device 170 (FIG. 17) and passed through the access sheath device 110. Using the suturing device, the suture would be attached to the middle of a double sided needle. The needle would be shuttled back and forth with subsequent passes of the suture through the tissue. After each pass through the tissue, the suturing device will grab the needle and hold the needle in position while the needle is passed through tissue on the opposite side.

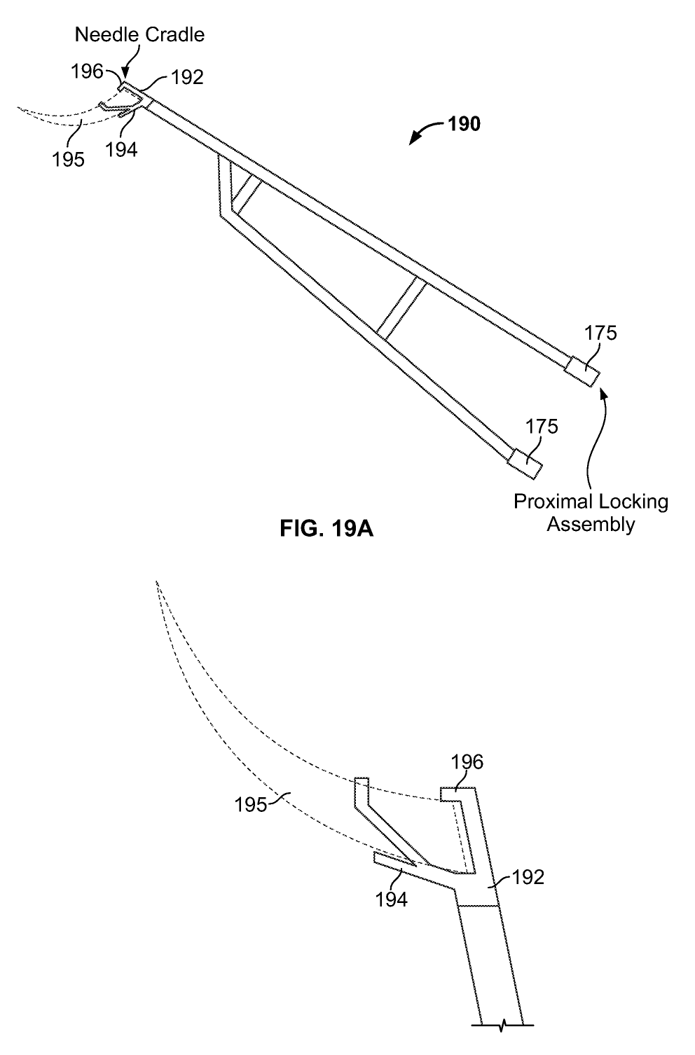

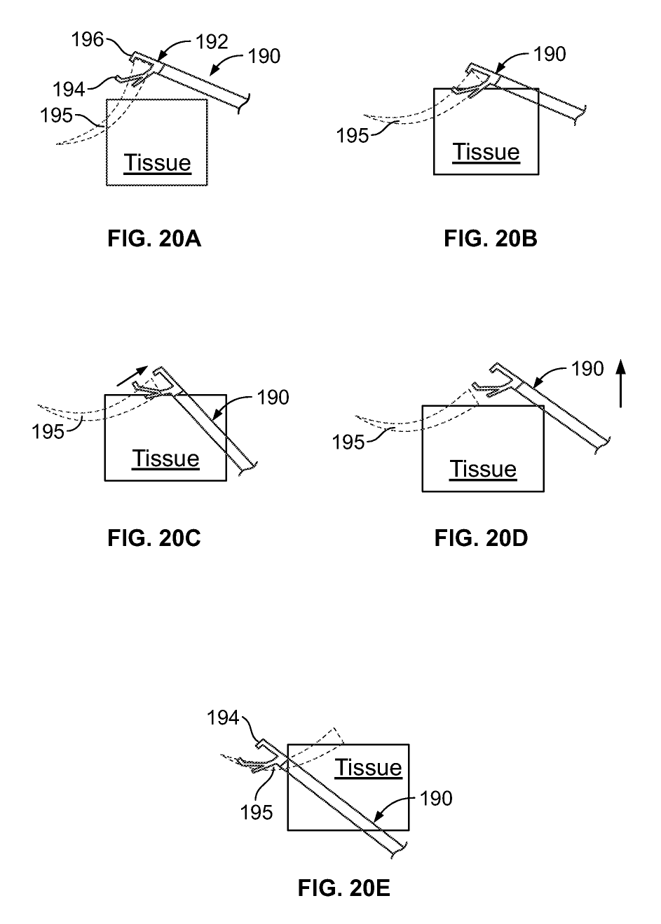

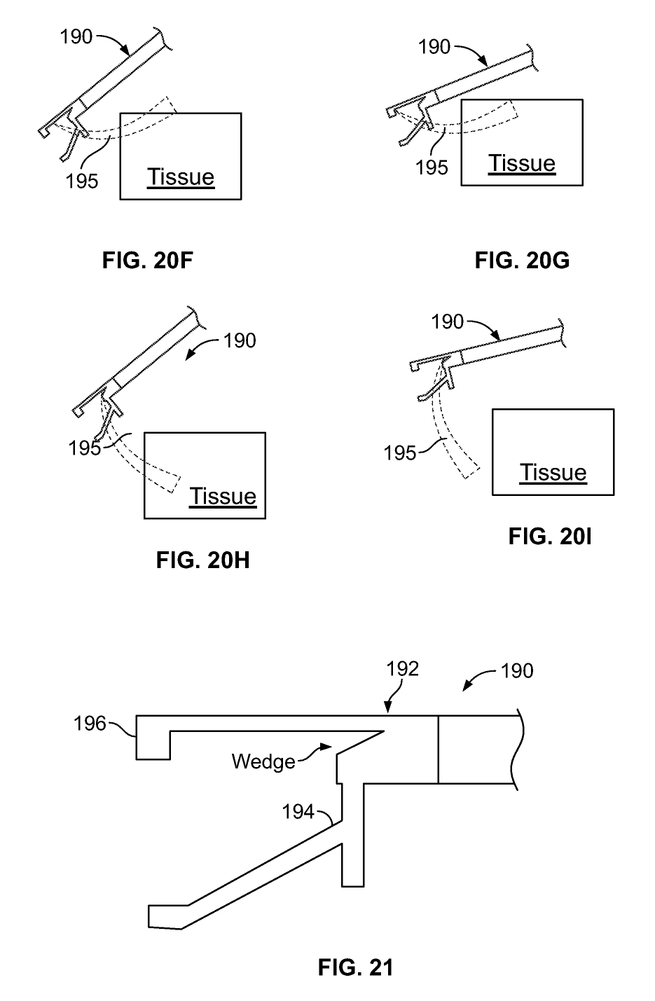

Referring to FIGS. 19A-B, FIGS. 20A-I, and FIG. 21, some embodiments of an intracavitary needle driver 190 can be utilized in the peritoneal space. The needle driver 190 can be attached to the interchangeable instrument holder device 170 (FIG. 17) and passed through the access sheath device 110. The needle driver 190 may include a needle cradle 192, distal needle control mechanism 194, and a proximal needle support system 196. In use, a needle 195 would be place into the cradle 192 with the swedged end of the needle engaging the proximal needle support system 196. Using the interchangeable instrument holder 170, the needle 195 would be placed into the target tissue (see, for example, FIGS. 20A-C). The needle driver 190 would then be freed from the needle (see, for example, FIG. 20D). To finish the needle throw, the needle driver 190 would then be used to grasp the needle 195. This would be accomplished by grasping the needle 195 using the distal needle control mechanism 194, rotating the needle driver 190, placing the tip of the needle 195 in the needle cradle 192, and pulling the needle 195 through the tissue (see, for example, FIGS. 20E-I). The needle 195 and suture would then be brought out from the target tissue and through the urethra to rest outside of the body 10. Using the other needle of the two armed suture, the same procedure would be performed at the targeted tissue. The suture would then be tied using a suture pushing device (not shown in FIGS. 19A-B, FIGS. 20A-I, and FIG. 21). Such an intracavitary suture pushing device may be used in conjunction with working port 177 of the interchangeable instrument holder 170 (FIG. 17). Both ends of the suture would be held externally as the suture pushing device is used to place a knot in position at the target tissue. The suture pushing device would be comprised on a metallic substance or a polymer. The suture pushing device will be based on modified designs proposed by the intervention or using commercially available designs.

In some embodiments, intracavitary biopsy forceps may be utilized in the peritoneal space. For example, the biopsy forceps may operate through the working channel of endoscope devices passed the working channel 177 of the interchangeable instrument holder 170 and passed through the access sheath device 110.

In some embodiments, an intracavitary cautery device may be utilized in the peritoneal space. The intracavitary cautery may be deployed in number of different manners. For example, the intracavitary cautery device may be deployable via attachment with the interchangeable instrument holder 170 (FIG. 17), via reducers using one or more inner sheath devices 150 (FIG. 11), via direct insertion through the access sheath device 110, or via endoscope devices passed through the access sheath device 110.

In some embodiments, an intracavitary stapling device may be utilized in the peritoneal space. The stapling device may be deployable via direct insertion through the access sheath device 110 or via one or more inner sheath devices 150.

In some embodiments, an intracavitary clip applier may be utilized in the peritoneal space. The clip applier may be a reusable device used directly or in conjunction with the interchangeable instrument holder 170 (FIG. 17). In one example, the clip applier can hold the clip at 3 separate points. The clip applier may be used to close the clip and thereby fix the clip upon the target tissue. The clip applier may be a substantially a rigid instrument.

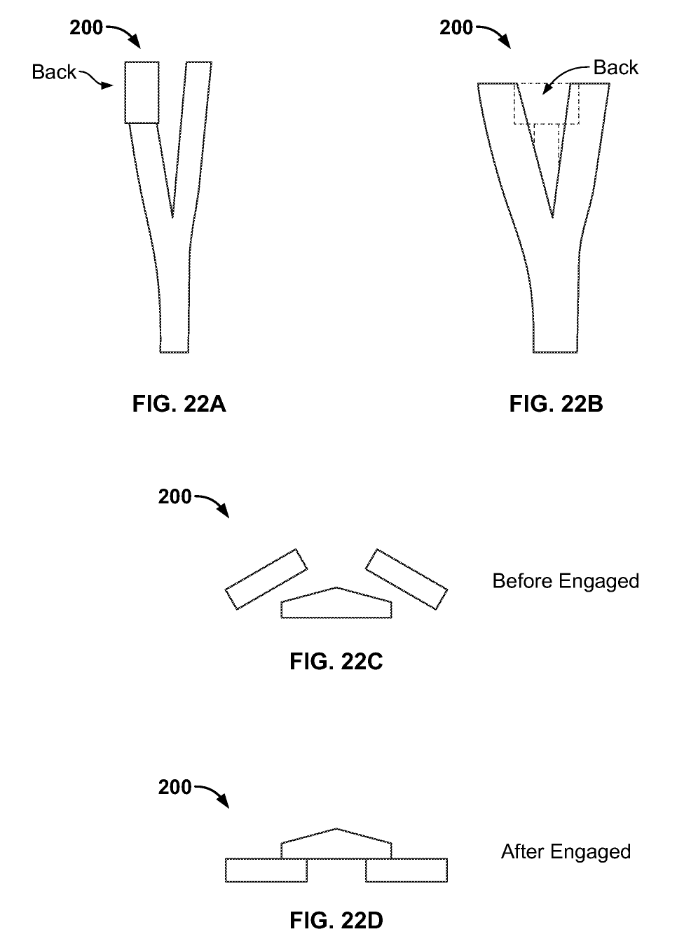

Referring to FIGS. 22A-D, some embodiments of intracavitary clips 200 may be utilized in the peritoneal space. The clips may be used with the previously described intracavitary clip applier. As shown in FIGS. 22A-D, the clips may comprise a unique three prong design. In some implementations, the clips may be used on an individual basis and be comprise a polymer material. In some embodiments, the polymer material may be biodegradable so that the clips degrade over time.

In some embodiments, an intracavitary specimen retrieval system may be utilized in the peritoneal space. The specimen retrieval system may include components such as a pleated bag and deployable bag holder/handle system. The specimen retrieval system may be used in a number of situations and in conjunction with or without the use of endoscope devices. In some embodiments, the pleated bag used for specimen retrieval may comprise a polymer sheet material or mesh and/or net design. Use of a mesh or net design may facilitate morcellation of the specimen alone and decrease likelihood that the specimen retrieval system would also be morcellated.

In some embodiments, an intracavitary specimen morcellation device may be utilized in the peritoneal space. The specimen morcellation device may operate similar to a tissue morcellation system used for transurethral surgery. The morcellation equipment may employ rigid equipment, however, as previously described, the use of rigid instruments is permissible for the transvesical peritoneal interventions described herein. In use, the tissue targeted for morcellation can be place in the intracavitary specimen retrieval system. The retrieval system (carrying the specimen) would then be maneuvered into the bladder 60. In some circumstances, morcellation can take place exclusively in the bladder as a safety precaution. The morcellation system can include a rotational cutting blade and a suction system. The suction system can draw the targeted tissue into contact with the rotational cutting blade. When using a suction based system, the preferred retrieval bag would be comprised of a mesh and/or net design (as previously described), thereby limiting the risk of the retrieval bag from being drawn into the rotational cutting system with suction.

In some embodiments, an intracavitary injector/applicator/hollow needle/suction and/or probe insertion assembly may be utilized in the peritoneal space. This multipurpose instrument can be placed with or without the use of the access sheath device 110 and with or without use of previously described inner sheath devices 150. The instrument may also be deployable directly through the interchangeable instrument holder 170. A number of functions can be accomplished with this device, including intracavitary suction, irrigation, application of substances within the target cavity, removal of specimens from the target cavity, and the ability to perform core tissue biopsy.

In some embodiments, an intracavitary hemostatic compression systems and/or tamponade systems may be utilized in the peritoneal space. During the transvesical interventions described herein, an unexpected hemorrhage may be encountered. In some circumstances, direct compression of the bleeding site may be warranted to assist in control of the hemorrhage or to control bleeding temporarily during the process of an emergent open conversion. The hemostatic compression system may include one or more inflatable balloons that could increase compressive force and/or surface area that may assist with vascular control. The balloon configuration may be selected depending upon the cavity in which bleeding was occurring. Likewise, non-emergent bleeding may occur during the normal course of an intervention performed in the peritoneal cavity. In this situation, introduction of a tamponade system will be included in the intervention. The tamponade system may comprise a highly absorbent material and an applicator. The systems would be deployed via the access sheath device 110, via the inner sheath device 150, or via the working port 177 of the interchangeable instrument holder device 170.

In some embodiments, an intracavitary hemostat or clamp may be utilized in the peritoneal space. Given the nature of intraperitoneal surgery, occasion may arise when need for a hemostatic type device or hemostatic clamp occurs. In this situation, deployment of an intracavitary hemostat or clamp may be required. The presently proposed device would be spring loaded approximately 2 cm in length and approximately 5 mm in diameter, however, for select indications the size of the clamps can be modified. The device may be deployed via the access sheath device 110 (or through an inner sheath device 150 disposed in the access sheath device 110). An applicator device for the deployable intracavitary hemostats may be a rigid instrument. In alternative embodiments, the applicator device may comprise a flexible instrument. The hemostat applicator may be inserted via the access sheath device 110 (or through an inner sheath device 150 disposed in the access sheath device 110).

In some embodiments, an intracavitary tool and materials container (“intraperitoneal toolbox”) may be positioned inside the peritoneal cavity for use during a number of interventions in the peritoneal cavity. For a given intracavitary intervention, a surgeon may use a plurality of smaller devices (e.g., clips, needles, or the like) to successfully perform the intervention. The intraperitoneal toolbox may contain the materials positioned in a novel system to facilitate flow of the intervention. The toolbox may be deployed via the access sheath device 110 (or through an inner sheath device 150 disposed in the access sheath device 110). For instance, the toolbox may hold suture and hemostatic clips to assist with the procedure. If an additional suture or clips were required during the intervention, the additional suture and/or clips could be removed from the toolbox rather than removing the intraperitoneal instruments or endoscope device.

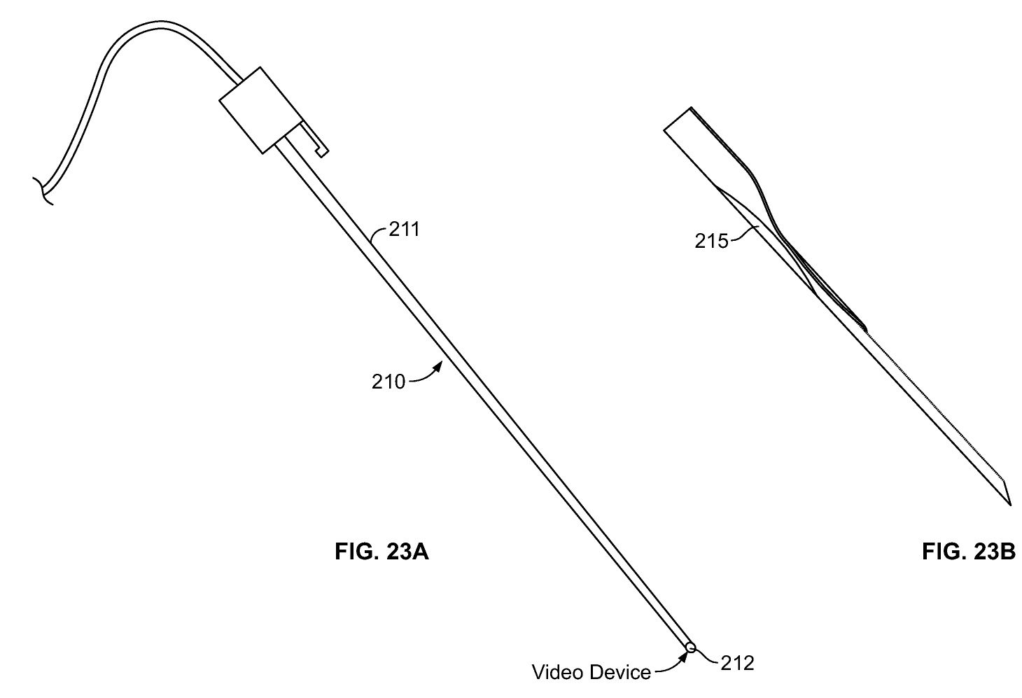

Referring to FIGS. 23A-B, some embodiments of a remote view imaging system 210 (e.g., “needle view” instruments) may be utilized during a transvesical intervention. For example, if the maximum working space through the access sheath device 110 is occupied by other devices, visualization of the surgical field may be provide using video chip technology (e.g., an image capturing device) on a needle view instrument 210. In this scenario, a small video chip 212 may be mounted to a tip mechanism of a visualization device 211. The visualization device 211 may be placed within a needle 215 (FIG. 23B). The needle 215 may be of sufficient length to penetrate the thickness of the abdominal wall. After entering the peritoneal cavity, the visualization device 211 may be lowered into position. To prevent complications with the needle 215, the needle 215 may then be retracted and snapped into the needle holding device. Images from the needle view instrument 210 may be viewed as the other instruments were used for transvesical peritoneal interventions.

In some embodiments, an intracavitary drainage catheter device may be utilized in the peritoneal space in the form of a transvesical intraperitoneal closed surgical drainage system with closed suction capability. In multiple interventions within the peritoneal cavity, a postprocedural drain may be required after an examination, therapy, or surgical intervention. Using transvesical intraperitoneal surgery, drain placement may be warranted in keeping with the principles of natural orifice surgery. The intraperitoneal drain can be placed in the peritoneal cavity with an exit point via the bladder 60 and urethra 70. The drain would be placed through the bladder incision 66 (FIG. 2) used to access the peritoneal cavity. The drain would be secured with suture to the Foley catheter draining the bladder after the transvesical peritoneal intervention. Depending on the planned intraperitoneal intervention, surgical drains may be in place for 2-5 days after the intervention.

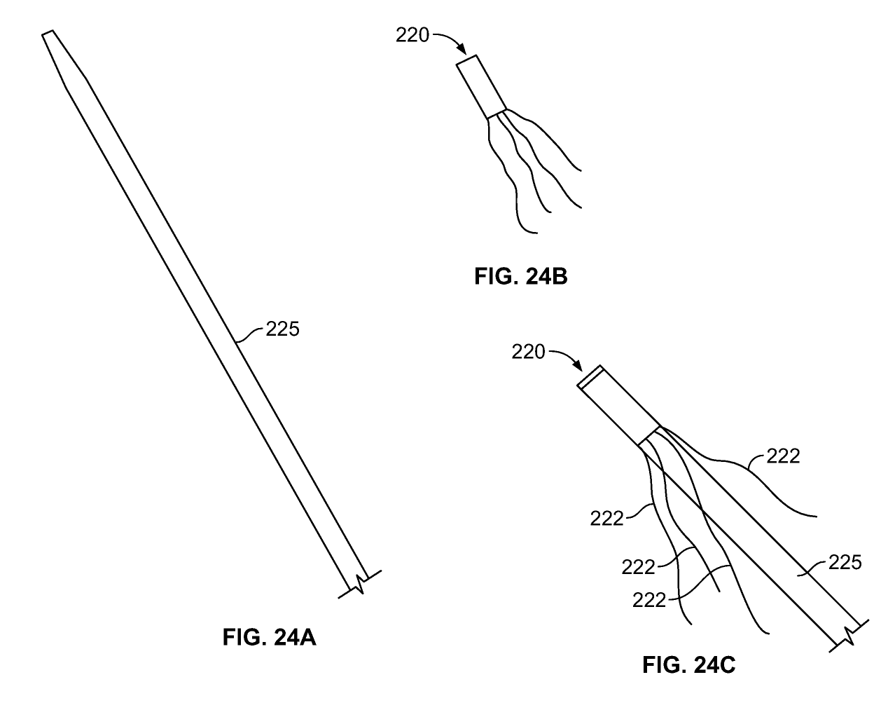

Referring to FIGS. 24A-C, some embodiments of an endoscopic holster apparatus 220 may be utilized in the peritoneal space to increase the maneuverability and flexibility of the endoscope devices 225. The endoscopic holster 220 may slide over the tip of the endoscope device 225. The endoscopic holster 220 may comprise a snug fitting polymer construction that can circumferentially cover approximately the distal 1 cm of the endoscope devices 225 (see, for example, FIG. 24C). The tip portion of the endoscope devices 225 need not be covered and the visualization system and working ports are not impeded. Steering lines 222 may extend from the holster 220. These steering lines 222 can be approximately double the length of the endoscope devices 225 being utilized. Two steering lines 222 may be located on each side of the holster. In situation when the endoscope devices 225 has unidirectional flexion, the contralateral steering lines 222 can be removable. When additional flexion of the endoscope devices 225 is required, the surgeon may grasp the steering lines 222 attached to the endoscopic holster 220 and provide gentle traction to facilitate increased flexion of the endoscope devices 225. Simultaneous fluoroscopic visualization during flexion can substantially prevent the steering lines 222 from overflexing the endoscope devices 225. From the description herein, it should be understood that use of an endoscopic holster apparatus 220 would not be limited to transvesical intraperitoneal interventions. For example, the endoscopic holster device 220 could be used to increase functionality and maneuverability of any one of a number of endoscope devices employed with or without transvesical intraperitoneal procedures.

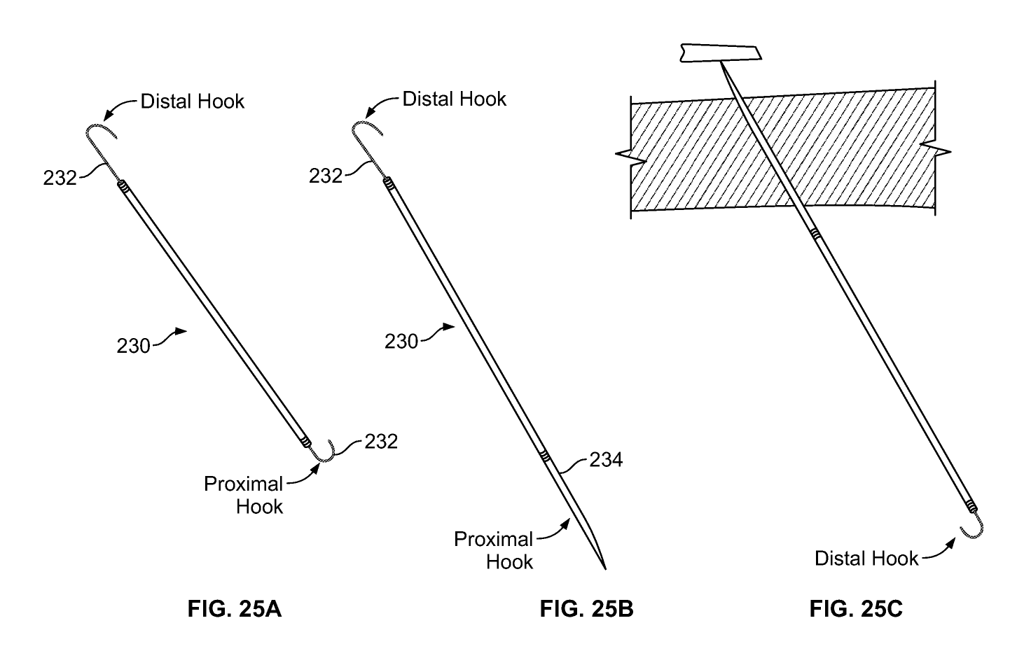

Referring to FIGS. 25A-C, some embodiments of intracavitary retraction devices 230 may be utilized in the peritoneal cavity. During the transvesical interventions described herein, some situations may exist where retraction of tissue is warranted for successful completion of the intraperitoneal procedure. In these circumstances, the intracavitary retraction devices 230 may be utilized. The retraction devices 230 can be deployed in the peritoneal space using attachments with the interchangeable instrument holder 170 or using traditional laparoscopic instrumentation. In one embodiment, the retraction system would include tissue hooks 232 on either end that would be attached to a stretchable piece of polymer (see, for example, FIG. 25A). For deployment of the retractor device 230, the proximal end would be hooked into the abdominal wall and the distal end would be hooked into the targeted structure. In other embodiments, the center portion of the retractor may be comprised of a coil, spring, or other configuration made of a variety of materials including metals or polymers. Another embodiment of a retraction device 230 would similarly have a hook 232 on the distal end that would be hooked into the target tissue. On the proximal end, however, the hook design would be replaced with a long needle 234 fixed to the center portion of the retractor device 230 (see, for example, FIG. 25B). With this design, the proximal end of the retractor device 230 would be pushed through the abdominal wall and an external clamp used to secure this end for retraction (FIG. 25C).

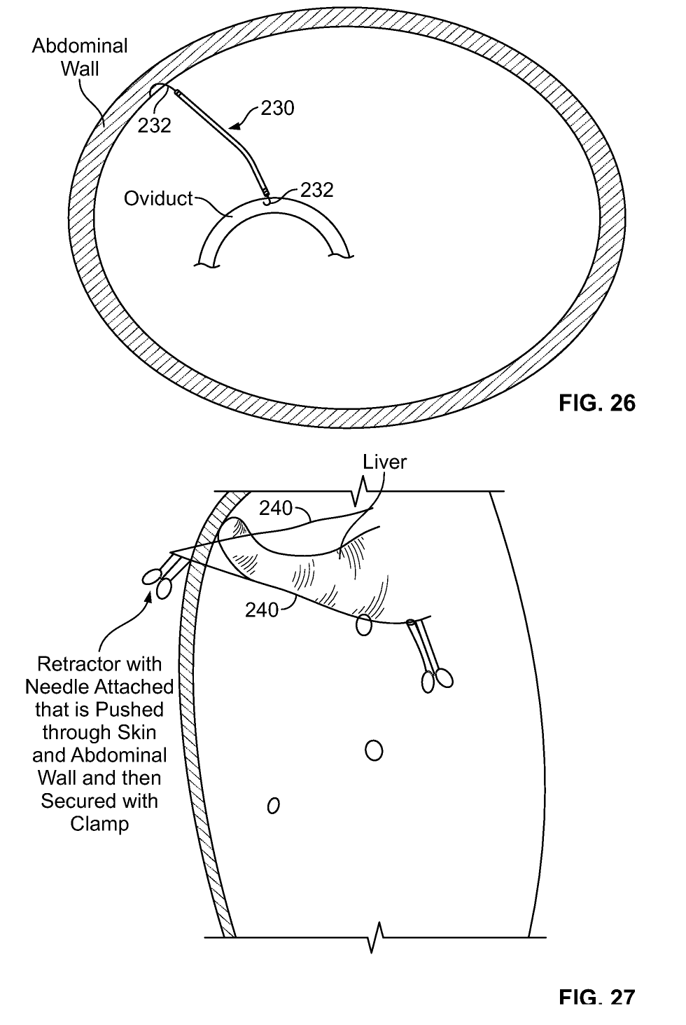

Referring to FIG. 26, one exemplary use of the retractor device 230 may be adjustment of the oviduct away from surrounding intraperitoneal contents during proposed transvesical intraperitoneal tubal ligation performed with clips. As a first step, the oviduct would be freed from surrounding tissues. The freed oviduct could then be retracted with the retraction device to provide ease of clipping. As such, the second step would be to place the retractor. This would elevate the oviduct in preparation for clip placement. The third step would be placement of the clip. The fourth step would be removed of the retractor.

Another example for the retraction device 230 would be during transvesical intraperitoneal cholecystectomy. The hook could be placed via the connective tissues of the gall bladder to facilitate dissection. The retractor device 230 would provide countertraction to simplify the dissection process.

The retraction device 230 may also be useful when making an opening in bowel or performing a bowel anastamosis during a transvesical intraperitoneal procedure. In either of these situations, the hook could be positioned into the mesentery of the bowel and similarly provide countertraction as the work was being performed.

Referring to FIG. 27, some embodiments of a retraction device 240 are not limited to use in transvesical intraperitoneal procedures. For example, when retraction is warranted during a laparoscopic procedure via the abdominal wall, the retractor device 240 could be deployed via the existing trocars to be an internal retraction system. In some embodiments, the retractor device 240 can be deployed internally with the proximal and distal ends being held in or out of the body. As such, the proposed retraction device 240 may also have utility during laparoscopic and robotic procedures. For example, as shown in FIG. 27, the retractor device 240 can be utilized for liver retraction during right sided laparoscopic or robotic procedures on the kidney. The proposed retraction system 240 would achieve liver retraction without the need for additional trocar placement. Another example for use of the retractor device 240 is when performing laparoscopic or robotic pyeloplasty. The retraction device 240 could be deployed such that the cut edge of the ureter is held on tension to facilitate performance of suturing.

Referring again to the previously described transvesical peritoneal approach, a number of surgical procedures can be performed on one or more intraperitoneal organs, such as biopsy procedures, appendectomy, cholecystectomy, stone surgery, fallopian tube occlusion with clips, lysis of adhesions, other solid organ removal procedures, and reconstructive procedures including procedures for obesity. Using this access technique, in short, an effective platform for an unlimited number of intraabdominal procedures in multiple subspecialties not limited to general surgery, gynecology, urology, and colorectal surgery could be performed.

Once the transvesical peritoneal intervention is finished, the endoscope devices and inner sheath devices 150 can be removed first. The second optics system 130 may then be reinserted into the access sheath device 110. Under direct vision, the access sheath device 110 may then be removed from the bladder wall 65. The interchangeable instrument holder device 170 may then be positioned in the bladder 60 with the needle driver instrument or needle closure device and the bladder incision 66 can be closed. At the culmination of the procedure, all endoscopes can be removed. A large caliber Foley catheter may be place in the bladder 60. If an intraperitoneal drain was placed, the drain tubing will be fixed to the Foley catheter with suture.

### EXAMPLES OF TRANSVESICAL PERITONEAL INTERVENTIONS

A transvesical peritoneal approach can be used for a variety of interventions including evaluation of all abdominal organs, biopsy of all abdominal organs, and removal of select intraabdominal organs such as the gall bladder and appendix. Using the suturing devices described herein, reconstructive procedures will also be possible including correction of blockages in the urinary and gastrointestinal tract. Potentially, procedures on the vascular system may also be possible using this approach. In addition, obesity surgery will also be possible using this platform. Specific examples of transvesical peritoneal interventions that can be performed are described herein. Two example procedures are described: transvesical peritoneoscopy with diagnostic evaluation and biopsy and transvesical peritoneoscopic cholecystectomy.

For transvesical peritoneoscopy with diagnostic evaluation and biopsy, transvesical access may be performed as described above. For this intervention, the initial portion of the endoscopic procedure may be performed solely via the access sheath device 110. The diagnostic evaluation may be performed using rigid and flexible endoscopes with a goal of assessing the ability to systematically evaluate all intraperitoneal organs. Via the access sheath device 110, the specific endoscopes may be utilized: flexible cystoscope, rigid cystoscope, flexible ureteroscope, and rigid ureteroscope. For the biopsy portion of the procedure, a rigid inner sheath device 150 may first be placed through the access sheath device 110. The access sheath device 110 and inner sheath device 150 may be fixed to the external scaffolding system 160. Using a rigid ureteroscope, biopsy of the liver may be performed and the biopsied area will be fulgurated. After the intervention is complete, the access sheath device 110 may be removed under direct vision, and the interchangeable instrument holder 170 with needle driver attachment may be introduced to close the cystotomy.

Transvesical peritoneoscopic cholecystectomy may likewise be performed after the standard access procedure is performed. Using two flexible ureteroscopes via a dual channel rigid inner sheath device 150, access to the gall bladder may be performed. The procedure may be used in conjunction endoscopic grasping forceps and electrocautery placed through the endoscopic working ports. A dissecting hook electrode used in conjunction with the interchangeable instrument holder may be used to dissect the blood supply to the gall bladder and dissect the cystic duct. A clip applier attached to the interchangeable instrument holder may be used to apply clips to the vasculature and cystic duct. Division of the structures may be performed using the scalpel attachment with the interchangeable instrument holder. The freed gall bladder may be placed in the specimen retrieval bag and brought through the cystotomy and removed intact. The cystotomy may then be closed with suture using the interchangeable instrument holder with needle driver attachment and the suture knot pusher device.

A number of embodiments of the invention have been described. Nevertheless, it will be understood that various modifications may be made without departing from the spirit and scope of the invention. Accordingly, other embodiments are within the scope of the following claims.

What is claimed is:

1. A system for transvesical access to a targeted site in a peritoneal space, comprising:
a transvesical access sheath having a proximal end, a distal end, and a channel extending longitudinally therethrough; and
an entry mechanism having a tissue penetration surface, the entry mechanism being disposed on a distal portion of the access sheath,
wherein, when the distal portion of the transvesical access sheath is passed through the urethra and into the bladder, the tissue penetration surface is insertable into a wall of the bladder to form an opening to a peritoneal space.

2. The system of claim 1, wherein the channel of the transvesical access sheath provides fluid communication with the peritoneal space when the distal portion of the access sheath is at least partially advanced through the opening.

3. The system of claim 2, wherein the transvesical access sheath has a longitudinal length such that the proximal portion remains outside the urethra when the distal portion is passed through the urethra and into the bladder.

4. The system of claim 3, further comprising one or more instruments to advance from the proximal portion of the transvesical access sheath, through the channel, and out of the distal portion.

5. The system of claim 4, wherein the one or more instruments are advanced to one or more organs in the peritoneal space when the distal portion of the access sheath is at least partially advanced through the opening.

6. The system of claim 5, wherein the one or more instruments comprises at least one rigid endoscope device to advance through the transvesical access sheath to the peritoneal space.

7. The system of claim 1, wherein the entry mechanism comprises a blundt entry screw mechanism.

8. The system of claim 7, wherein the tissue penetration surface includes one or more tissue engagement threads to engage the bladder wall after penetrating into the peritoneal space.

9. The system of claim 7, wherein the blundt entry screw mechanism is operable to engages the bladder wall free of a sharp needle-like tip.

10. The system of claim 1, further comprising an access apparatus having at least one sheath holder device to releasably coupled with the transvesical access sheath.

11. The system of claim 7, wherein the access apparatus guides advancement of the transvesical access sheath when the transvesical access sheath is advanced through the urethra and into the bladder.

12. The system of claim 8, wherein the access apparatus includes a first optics system that video monitoring of the urinary tract when the transvesical access sheath is advanced toward the bladder wall.

13. The system of claim 9, further comprising a second optics system arranged within the channel of the transvesical access sheath so as to provide contemporaneous monitoring using both the first optics system and the second optics system d when the transvesical access sheath is advanced into the peritoneal space.

14. The system of claim 1, wherein at least the portion of the distal end of the transvesical access sheath comprises a substantially translucent or transparent material to provide visual monitoring of the bladder wall when the tissue penetration surface is inserted into the bladder wall to form the opening to the peritoneal space.

15. The system of claim 1, further comprising an instrument induction assembly that releasably mounts to the proximal end of the transvesical access sheath to provide a substantial seal at the proximal end of the transvesical access sheath.

16. The system of claim 1, further comprising at least one inner sheath to advance through the channel of the transvesical access sheath and into the peritoneal space.

17. The system of claim 16, wherein the at least one inner sheath comprises a flexible and steerable distal portion.

18. The system of claim 1, further comprising an external access instrumentation scaffolding system arranged in proximity to the proximal end of the transvesical access sheath.

19. The system of claim 1, further comprising an interchangeable instrument holder having a distal portion that advances through the channel of the transvesical access sheath, the distal portion of the interchangeable instrument holder being releasably attachable to two or more instruments selected from the group consisting of: a scalpel, scissors, a suturing device, a needle driver, a suture pushing device, biopsy forceps, a stapling device, a clip applier, a specimen retrieval device, a specimen morcellation device, and an intracavitary injector probe assembly.

20. A method for transvesical access to a targeted site in a peritoneal space, comprising:
passing a transvesical access sheath through the urethra and into the bladder of the urinary tract;
forming an opening in a wall of the urinary tract proximate to a peritoneal space; and
inserting at least a portion of the transvesical access sheath through the opening in wall of the urinary tract so that a channel extending through the transvesical access sheath is in communication with the peritoneal space.

21. The method of claim 20, wherein the opening in the wall of the urinary tract is formed through a wall of a bladder adjacent the peritoneal space.

22. The method of claim 21, further comprising providing a direct line of site from a proximal portion of the transvesical access sheath, through the channel, and to one or more organs in the peritoneal space.

23. The method of claim 20, further comprising accessing the peritoneal space without obstructing a patient's airway with an instrument.

24. The method of claim 20, further comprising advancing a rigid endoscope device through the transvesical access sheath to the peritoneal space.

25. The method of claim 20, further comprising advancing a flexible endoscope device through the transvesical access sheath to the peritoneal space.

26. The method of claim 20, wherein the inserting step occurs without caustic gastric contents to spilling into the peritoneal space.

27. The method of claim 20, further comprising examining the external surface of stomach with one or more instruments advanced through the transvesical access sheath.
